(19) United States
(12) Patent Application Publication
(10) Pub. No.: US 2022/0411755 A1
(43) Pub. Date: Dec. 29, 2022

(54) HEMATOPOIETIC PRECURSOR CELL PRODUCTION

(71) Applicant: AMGEN INC., Thousand Oaks, CA (US)

(72) Inventors: Stuart CHAMBERS, Daly City, CA (US); Jingli Hu ZHANG, Pleasanton, CA (US); Qingwen CHENG, Palo Alto, CA (US); Guanyi HUANG, Foster City, CA (US)

(73) Assignee: AMGEN INC., Thousand Oaks, CA (US)

(21) Appl. No.: 17/776,622

(22) PCT Filed: Nov. 13, 2020
(86) PCT No.: PCT/US2020/060582

§ 371 Date: May 13, 2022

Related U.S. Application Data
(60) Provisional application No. 62/935,353, filed on Nov. 14, 2019.

(51) Int. Cl. C12N 5/0789

(52) U.S. Cl. CPC C12N 5/0647; C12N 2500/02; C12N 2500/99; C12N 2501/115; C12N 2501/155; C12N 2501/41; C12N 2506/45

(57) ABSTRACT

This invention provides improved methods for generation of hematopoietic precursor cells from a pluripotent stem cell and hematopoietic precursor cells generated thereof. The hematopoietic precursor cells express CXCR4 or runx1c and are capable of homing and/or engraftment in bone marrow.

### FIELD OF THE INVENTION

The present invention relates to hematopoietic stem cell production with improved properties.

### BACKGROUND OF THE INVENTION

Hematopoietic cells or blood cells are in great demand for clinical applications and for laboratory use. In the clinic, hematopoietic stem cells (HSCs) can be used to reconstitute hematopoiesis in patients that have undergone a therapy that suppresses hematopoiesis, such as an anti-cancer therapy, or in patients that have inherited hematological diseases. In addition, red blood cells, platelets, and neutrophil granulocytes can be used in blood transfusions and in the treatment of certain hematological disorders. In the lab, blood cells can be used for many applications including drug screening.

Currently, blood cells for such clinical and laboratory applications are obtained from living donors. However, the limited supply of donor blood, especially when a genetically-compatible donor is required, limits therapeutic applications and drug screening. Thus, there remains a need to develop sources of blood cells other than donor blood. For example, there is a need for an unlimited supply of well characterized functional blood cell types, including patient specific HSCs for therapeutic applications.

Myeloid cells originate from multipotent hematopoietic stem cells in the bone marrow and consist of granulocytes (neutrophils, eosinophils, basophils) and cells of monocyte/macrophage lineage including dendritic cells (DCs) and osteoclasts. These cells play a critical role in innate and adaptive immunity, inflammatory reactions, and bone remodeling.

We have established human pluripotent stem cell (hPSCs) differentiation protocol to generate hematopoietic stem cells (HSC). Hematopoiesis occurs in two phases during embryonic development—the primitive phase and definitive phase. Definitive hematopoiesis is characterized by generation of long-term repopulating HSCs with broad potential for cell therapy and disease modeling, which have not been previously obtained from hPSCs.

### SUMMARY OF THE INVENTION

The present invention is based, in part, on the discovery of a method of producing hematopoietic stem cells (HSCs) from inducible pluripotent stem cells (iPSCs). In some embodiments, the invention is a method of producing a hematopoietic precursor cell comprising the steps of:
a) obtaining a population of pluripotent stem cells;
b) culturing the cells on day 0 in supplemented serum-free differentiated (SFD) medium under a first hypoxic condition;
c) culturing the cells in StemPro-34 medium under a second hypoxic condition;
d) culturing the cells in StemPro-34 medium under non-hypoxic conditions; and
e) culturing the cells in StemPro-34 medium under non-hypoxic expansion conditions; and
f) collect population of hematopoietic precursor cells.

In one embodiment, the method of producing a hematopoietic precursor cell from a pluripotent stem cell or transdifferentiation of a somatic cell, comprises culturing the pluripotent stem cell or somatic cell under conditions to generate the hematopoietic precursor cell that can differentiate into different hematopoietic lineage cells, comprising the steps of (a) obtaining a population of pluripotent stem cells, (b) inducing hematopoietic differentiation by culturing on day 0 in SFD medium, 10 μM Y-27632, 10 ng/ml BMP4 and 25 ng/ml bFGF; culturing for 1-2 days with SFD medium, 10 ng/ml BMP4, 5 ng/ml bFGF, and 8 uM CHIR99021; culturing for 1 day with StemPro34 medium, 12.5 ng/ml bFGF, and 25 ng/ml VEGF; culturing for 1-2 day with StemPro34 medium, 12.5 ng/ml bFGF, and 25 ng/ml VEGF; culturing for 2-4 days with StemPro34 medium, 12.5 ng/ml bFGF, 25 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO; culturing for 3-5 days with StemPro34 medium, 12.5 ng/ml bFGF, 12.5 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, 2 U/ml EPO, 10 ng/ml BMP4, 10 ng/ml SHH, 10 ug/ml Angiotensin II, and 100 uM Losartan potassium, replaced each day; culturing for 5-10 days with StemPro34 medium, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO replaced every 3 days.

In some embodiments, the invention is a hematopoietic precursor cell, such as a hematopoietic stem cell, produced using the above method. In some preferred embodiments, the hematopoietic precursor cell expresses CXCR4 on the cell surface. In some embodiments, the hematopoietic precursor cell is CD34+, CD45+, CD90+, or THY1+. In some embodiments, the hematopoietic precursor cell CD38−, Lin−, CD43−, and CD73−. In some embodiments, the hematopoietic precursor cell expresses CD90 on the cell surface. In some embodiments, the hematopoietic precursor cell expresses runx1c. In some preferred embodiments, the hematopoietic precursor cell is capable of generating long-term repopulating hematopoietic precursor cells.

### BRIEF DESCRIPTION OF THE FIGURES

FIG. 1 shows FACS plots showing hemogenic endothelium formation from iPSC, using an earlier protocol as well as the protocol shown in Example 1.

FIG. 2 shows generation of HSC-like cells from iPSC-derived hemogenic endothelium cells at day 21.

FIG. 3 shows the results of a limiting dilution assay at day 21 of differentiation.

FIG. 3A shows the percent of the wells having each cell type when the wells were loaded with a different number of cells. FIG. 3B shows the number of colonies formed of different cell types following loading by a different number of cells.

FIG. 4 shows the generation of GFP-2A-Runxlc hiPSC reporter line for labeling of hematopoietic stem cells (HSCs). FIG. 4A shows a schematic picture showing the strategy to target Runxlc genomic locus: Runxlc is transcribed from the distal promoter with a unique exon. Guide RNA was designed to specifically target the ATG start codon of Runxlc transcript for precise genome editing. A GFP-2A sequence was fused at the N-terminus to fluorescently label Runxlc positive hematopoietic stem cells during differentiation. The LoxP-PGK-BSD-pA-LoxP selection cassette was place in intron 1 to facilitate enrichment of correctly targeted cells populations. PCR primers (see Table 1) were designed to amplify the left junction of homologous recombination and the GFP-2A-Runxlc linker sequence. FIG. 4B shows that the primers described in 4A was used for screening positive colonies after genome editing. After blasticidin selection, a total of 48 single cell clones were picked, expanded and subjected PCR genotyping analysis. 38 clones exhibited positive genotyping band on agarose gel (efficiency=79%). FIG. 4C shows an image of the selected positive clone of GFP-2A-Runxlc hiPSC line.

FIG. 5 shows a visualization of GFP positive hematopoietic stem cells in hiPSC differentiation: GFP-2A-Runxlc iPSCs (d0, top left panel) were firstly differentiated into endothelium (d9, top right panel), followed by induction of endothelial-hematopoietic transition (EHT) that results in emergence of GFP positive hematopoietic stem cells (d14, mid panel) from selected regions (dashed box, “blood island”) of GFP negative endothelial layer. At day 17, the production of GFP positive HSCs are no longer restricted in certain regions, but became more prominent throughout the tissue culture (d17 bottom panel).

FIG. 6 shows a time course of surface marker expression pattern of GFP-2A-Runx1c iPSCs during hematopoietic differentiation: (A) Single positive population. (B) Runx1c+CD34+CD45+ putative hematopoietic stem cell population.

FIG. 7 shows HSC CD34 vs GFP-Runx1c expression on days 9 and 14.

FIG. 8 shows HSC CD34 vs GFP-Runx1c expression on days 16 and 17.

FIG. 9 shows HSC CD34 vs GFP-Runx1c expression on days 20 and 21.

FIG. 10 shows cell population sorting for CFU assays from LT-iPSC and GFP-Runx1c iPSC.

FIG. 11 shows CFU total cell counts on days 16, 17, 20, and 21.

FIG. 12 shows a CFU panel of common progenitor markers on days 16, 17, 20, and 21.

FIG. 13 shows a CFU panel of lymphoid progenitor markers on days 16, 17, 20, and 21.

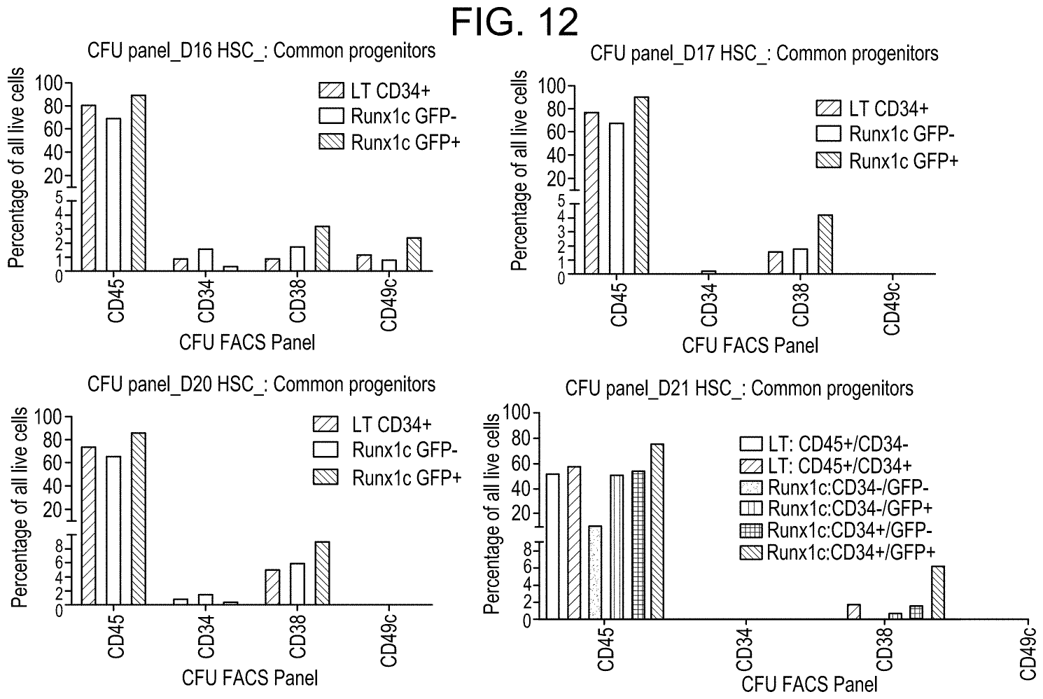

FIG. 12 shows a CFU panel of myeloid progenitor markers on days 16, 17, 20, and 21.

### DETAILED DESCRIPTION OF THE INVENTION

Recently, pluripotent stem cell lines have been obtained from human fibroblasts through insertion of certain genes critical for the maintenance of pluripotency of hESCs (Yu, J., et al. 2007, Science. 318:1917-1920. Takahashi, K., et al. 2007, Cell. 131:861-872. Park, I. H., et al. 2008, Nature. 451:141-146.). These so-called human induced pluripotent stem cells (iPSCs) behave similarly to hESCs, i.e., they are capable of self-renewal and large-scale expansion and differentiation toward all three germ layers. The hope is that iPSC lines generated from patients with various diseases could be used to obtain any type of progenitor or differentiated cell carrying a particular genetic trait at the cellular level, thus providing a unique opportunity to analyze disease pathogenesis in vitro.

Previously, a system was established for hematopoietic differentiation of hESCs into hematopoietic cells through coculture with OP9 bone marrow stromal cells (Vodyanik, M. A., Bork, J. A., Thomson, J. A., Slukvin, 1.1. 2005, Blood. 105:617-626) and characterized the two subpopulations of the most primitive multipotent hematopoietic cells to appear in OP9 cocultures of hESCs on the basis of their common expression of CD43 and differential expression of CD45. The lin-CD34+CD43+CD45− cells with broad lymphomyeloid differentiation potential appear first in coculture. Later, lin-CD34+CD43+CD45+ cells enriched in myeloid progenitors emerge (Vodyanik, M. A., Thomson, J. A., Slukvin, I. I. 2006, Blood. 108:2095-2105.). The Slukvin lab demonstrated that a similar pattern of hematopoietic differentiation is observed when iPSCs differentiate into blood cells in coculture with OP9 (Choi, K., et al. 2009, Stem Cells. 27:559-567.).

In certain embodiments of the invention, there are disclosed methods and compositions for providing hematopoietic cells or precursors of hematopoietic cells by forward programming of human pluripotent cells that are not hematopoietic cells, including stem cells, which includes human embryonic stem cells and inducible pluripotent stem cells, or by transdifferentiation of somatic cells that are not hematopoietic cells. Also provided are cells that comprise exogenous expression cassettes including one or more hematopoietic precursor programming factor genes and/or reporter expression cassettes specific for hematopoietic cell or hematopoietic precursor cell identification. In some embodiments, the cells may be stem cells, including but not limited to, embryonic stem cells, fetal stem cells, or adult stem cells. In further embodiments, the cells may be any somatic cells.

Stem cells are cells found in most, if not all, multi-cellular organisms. They are characterized by the ability to renew themselves through mitotic cell division and the ability to differentiate into a diverse range of specialized cell types. The two broad types of mammalian stem cells are: embryonic stem cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, and also maintain the normal turnover of regenerative organs, such as blood, skin or intestinal tissues.

Human pluripotent stem cells (including Human embryonic stem cells (ESCs) and induced pluripotent stem cells (iPSCs)) are capable of long-term proliferation in vitro, while retaining the potential to differentiate into all cell types of the body, including hematopoietic cells and hematopoietic precursor cells. Thus, these cells could potentially provide an unlimited supply of patient-specific functional hematopoietic cells and hematopoietic precursor cells for both drug development and therapeutic uses. The differentiation of human ESCs/iPSCs to hematopoietic cells and hematopoietic precursor cells in vitro recapitulates normal in vivo development; i.e. they undergo the normal sequential developmental stages including mesoderm differentiation and hematopoietic specification. That sequential developmental process requires the addition of different growth factors at different stages of differentiation. Certain aspects of the invention provide fully functional hematopoietic precursor cells by forward programming from human ESCs/iPSCs or transdifferentiation from somatic cells via expression of a combination of transcription factors important for hematopoietic cell differentiation/function, similar to the generation of iPSCs, bypassing most-if not all-normal developmental stages. This approach may be more time- and cost-efficient, and generate hematopoietic precursor cells and hematopoietic cells with functions highly similar, if not identical, to human adult hematopoietic cells and precursors of hematopoietic cells. In addition, human ESC/iPSCs, with their unlimited proliferation ability, may be advantageous over somatic cells as the starting cell population for hematopoietic precursor cell differentiation. Examples of hematopoietic cells and precursors of hematopoietic cells produced as part of the invention include cells expressing CXCR4, cells that are CD34+, CD45+, CD90+ and THY1+, cells that are CD38−, Lin−, CD43− or CD73−, cells that are CD45+, CD34+, CD90+, CD38−, and Lin−, cells expressing CD90, cells expressing runx1c, or any combination of the above.

Embryonic stem cell lines (ES cell lines) are cultures of cells derived from the epiblast tissue of the inner cell mass (ICM) of a blastocyst or earlier morula stage embryos. A blastocyst is an early stage embryo-approximately four to five days old in humans and consisting of 50-150 cells. ES cells are pluripotent and give rise during development to all derivatives of the three primary germ layers: ectoderm, endoderm and mesoderm. In other words, they can develop into each of the cell types of the adult body when given sufficient and necessary stimulation for a specific cell type. They do not contribute to the extra-embryonic membranes or the placenta.

Most research to date used mouse embryonic stem cells (mES) or human embryonic stem cells (hES). Both have the essential stem cell characteristics, yet they require very different environments in order to maintain an undifferentiated state. Mouse ES cells may be grown on a layer of gelatin and require the presence of Leukemia Inhibitory Factor (LIF). Human ES cells could be grown on a feeder layer of mouse embryonic fibroblasts (MEFs) and often require the presence of basic Fibroblast Growth Factor (bFGF or FGF-2). Without optimal culture conditions or genetic manipulation (Chambers et al., 2003), embryonic stem cells will rapidly differentiate.

A human embryonic stem cell may also be defined by the presence of several transcription factors and cell surface proteins. The transcription factors Oct-4, Nanog, and Sox-2 form the core regulatory network that ensures the suppression of genes that lead to differentiation and the maintenance of pluripotency (Boyer et al., 2005). Cell surface antigens commonly used to identify hES cells include the glycolipids SSEA3 and SSEA4 and the keratan sulfate antigens Tra-1-60 and Tra-1-81.

Human ES cells can be obtained from blastocysts using previously described methods (Thomson et al., 1995; Thomson et al., 1998; Thomson and Marshall, 1998; Reubinoff et al, 2000.) In one method, day-5 human blastocysts are exposed to rabbit anti-human spleen cell antiserum, then exposed to a 1:5 dilution of Guinea pig complement to lyse trophectoderm cells. After removing the lysed trophectoderm cells from the intact inner cell mass, the inner cell mass is cultured on a feeder layer of gamma-inactivated mouse embryonic fibroblasts and in the presence of fetal bovine serum. After 9 to 15 days, clumps of cells derived from the inner cell mass can be chemically (i.e. exposed to trypsin) or mechanically dissociated and replated in fresh medium containing fetal bovine serum and a feeder layer of mouse embryonic fibroblasts. Upon further proliferation, colonies having undifferentiated morphology are selected by micropipette, mechanically dissociated into clumps, and replated (see U.S. Pat. No. 6,833,269). ES-like morphology is characterized as compact colonies with apparently high nucleus to cytoplasm ratio and prominent nucleoli. Resulting ES cells can be routinely passaged by brief trypsinization or by selection of individual colonies by micropipette. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as matrigel or laminin in the presence of “conditioned” medium containing basic fibroblast growth factor (Xu et al., 2001). The medium is previously conditioned by coculturing with fibroblasts.

Another source of ES cells are established ES cell lines. Various mouse cell lines and human ES cell lines are known and conditions for their growth and propagation have been defined. For example, the mouse CGR8 cell line was established from the inner cell mass of mouse strain 129 embryos, and cultures of CGR8 cells can be grown in the presence of LIF without feeder layers. As a further example, human ES cell lines Hl, H7, H9, H13 and H14 were established by Thompson et al. In addition, subclones H9.1 and H9.2 of the H9 line have been developed. It is anticipated that virtually any ES or stem cell line known in the art may be used with the present invention, such as, e.g., those described in Yu and Thompson (2008) Genes Dev 22(15):1987-97, which is incorporated herein by reference.

The source of ES cells for use in connection with the present invention can be a blastocyst, cells derived from culturing the inner cell mass of a blastocyst, or cells obtained from cultures of established cell lines. Thus, as used herein, the term “ES cells” can refer to inner cell mass cells of a blastocyst, ES cells obtained from cultures of inner mass cells, and ES cells obtained from cultures of ES cell lines.

Induced pluripotent stem (iPS) cells are cells that have the characteristics of ES cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., 2007). The transfected cells are plated on SNL feeder cells (a mouse cell fibroblast cell line that produces LIF) in medium supplemented with basic fibroblast growth factor (bFGF). After approximately 25 days, colonies resembling human ES cell colonies appear in culture. The ES cell-like colonies are picked and expanded on feeder cells in the presence of bFGF.

Based on cell characteristics, cells of the ES cell-like colonies are induced pluripotent stem cells. The induced pluripotent stem cells are morphologically similar to human ES cells, and express various human ES cell markers. Also, when grown under conditions that are known to result in differentiation of human ES cells, the induced pluripotent stem cells differentiate accordingly. For example, the induced pluripotent stem cells can differentiate into cells having hematopoietic cell structures and hematopoietic cell markers. It is anticipated that virtually any iPS cells or cell lines may be used with the present invention, including, e.g., those described in Yu and Thompson, 2008.

In another method, human fetal or newborn fibroblasts are transfected with four genes, Oct4, Sox2, Nanog and Lin28 using lentivirus transduction (Yu et al., 2007). At 12-20 days post infection, colonies with human ES cell morphology become visible. The colonies are picked and expanded. The induced pluripotent stem cells making up the colonies are morphologically similar to human ES cells, express various human ES cell markers, and form teratomas having neural tissue, cartilage, and gut epithelium after injection into mice.

Methods of preparing induced pluripotent stem cells from mouse are also known (Takahashi and Yamanaka, 2006). Induction of iPS cells typically require the expression of or exposure to at least one member from Sox family and at least one member from Oct family. Sox and Oct are thought to be central to the transcriptional regulatory hierarchy that specifies ES cell identity. For example, Sox may be Sox-1, Sox-2, Sox-3, Sox-15, or Sox-18; Oct may be Oct-4. Additional factors may increase the reprogranmiing efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Kif and, optionally, c-Myc.

iPS cells, like ES cells, have characteristic antigens that can be identified or confirmed by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews et al., 1987). Pluripotency of embryonic stem cells can be confirmed by injecting approximately 0.5-10×106 cells into the rear leg muscles of 8-12 week old male SCID mice. Teratomas develop that demonstrate at least one cell type of each of the three germ layers.

In certain aspects of the present invention, iPS cells are made from reprogramming somatic cells using reprogramming factors comprising an Oct family member and a Sox family member, such as Oct4 and Sox2 in combination with Kif or Nanog as described above. The somatic cell for reprogramming may be any somatic cell that can be induced to pluripotency, such as a fibroblast, a keratinocyte, a hematopoietic cell, a mesenchymal cell, a liver cell, a stomach cell, or a ˜ cell. In a certain aspect, T cells may also be used as source of somatic cells for reprogramming (see U.S. Application No. 61/184,546, incorporated herein by reference).

Reprogramming factors may be expressed from expression cassettes comprised in one or more vectors, such as an integrating vector or an episomal vector, e.g., an EBY element-based system (see U.S. Application No. 61/058,858, incorporated herein by reference; Yu et al., 2009). In a further aspect, reprogramming proteins could be introduced directly into somatic cells by protein transduction (see U.S. Application No. 61/172,079, incorporated herein by reference).

In certain aspects of the invention, there may also be provided methods of transdifferentiation, i.e., the direct conversion of one somatic cell type into another, e.g., deriving hematopoietic precursor cells or hematopoietic cells from non-hematopoietic somatic cells. However, human somatic cells may be limited in supply, especially those from living donors. In certain aspects, to provide an unlimited supply of starting cells for programming, somatic cells may be immortalized by introduction of immortalizing genes or proteins, such as hTERT or oncogenes. The immortalization of cells may be reversible (e.g., using removable expression cassettes) or inducible (e.g., using inducible promoters).

Somatic cells in certain aspects of the invention may be primary cells (non-immortalized cells), such as those freshly isolated from an animal, or may be derived from a cell line (immortalized cells). The cells may be maintained in cell culture following their isolation from a subject. In certain embodiments, the cells are passaged once or more than once (e.g., between 2-5, 5-10, 10-20, 20-50, 50-100 times, or more) prior to their use in a method of the invention. In some embodiments the cells will have been passaged no more than 1, 2, 5, 10, 20, or 50 times prior to their use in a method of the invention. They may be frozen, thawed, etc.

The somatic cells used or described herein may be native somatic cells, or engineered somatic cells, i.e., somatic cells which have been genetically altered. Somatic cells of the present invention are typically marmnalian cells, such as, for example, human cells, primate cells or mouse cells. They may be obtained by well-known methods and can be obtained from any organ or tissue containing live somatic cells, e.g., blood, bone marrow, skin, lung, pancreas, liver, stomach, intestine, heart, reproductive organs, bladder, kidney, urethra and other urinary organs, etc.

Mammalian somatic cells useful in the present invention include, but are not limited to, Sertoli cells, endothelial cells, granulosa cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macro phages, monocytes, mononuclear cells, cardiac muscle cells, and other muscle cells, etc.

Somatic cells may be partially or completely differentiated. Differentiation is the process by which a less specialized cell becomes a more specialized cell type. Cell differentiation can involve changes in the size, shape, polarity, metabolic activity, gene expression and/or responsiveness to signals of the cell. For example, hematopoietic stem cells differentiate to give rise to all the blood cell types including myeloid (monocytes and macrophages, neutrophils, basophils, eosinophils, erythrocytes, megakaryocytes/platelets, dendritic cells), erythro-megakaryocytic (erythrocytes, megakaryocytes, thrombocytes), and lymphoid lineages 10 (T-cells, B-cells, natural killer (NK) cells). During progression along the path of differentiation, the ultimate fate of a cell becomes more fixed. As described herein, both partially differentiated somatic cells and fully differentiated somatic 15 cells can be programmed as described herein to produce desired cell types such as hematopoietic cells and hematopoietic precursor cells.

In one embodiment, the present invention is a method to efficiently produce neutrophils, eosinophils, macrophages, osteoclasts, dendritic and Langerhans cells from mammalian pluripotent stem cells, preferably human embryonic stem cells (hESCs) or induced pluripotent stem cells (iPSCs, see, for example, Yu et al. (2007) Science 318:1917-1920, incorporated by reference, for one method of making iPSCs) through differentiation of the hESCs or iPSCs into lin-CD34+CD43+CD45+ myeloid-progenitors enriched cells using the described methods. In some embodiments, cells may further differentiate into lin+CD34-CD43-CD45+ progenitors.

### Generation of Lin-CD34+CD43+CD45+ Cell Population

The present invention is based, in part, on the discovery of a method of producing hematopoietic stem cells (HSCs) from human pluripotent stem cells (hPSC). The hPSC can be an inducible pluripotent stem cells (iPSCs), embryonic stem cell, or transdifferentiated somatic cell. The HSCs produced from the methods of the invention can differentiate into different hematopoietic lineage cells. The methods of the present invention comprise the following steps:

A first step is obtaining a cell or a population of human pluripotent stem cells (hPSC), which can be derived from embryonic stem cells, inducible pluripotent stem cells, or transdifferentiated somatic cells, as described above.

The next step is culturing the cells on day 0 in supplemented serum-free differentiated (SFD) medium (75:25 of IMDM:Ham's F-12, 0.05% BSA, 1×B27, 0.5×N2 supplements, 1× GlutaMax and 1× Penicillin-Streptomycin, 0.5 mM ascorbic acid, 450 μM Monothioglycerol, and 150 μg/mL holo-transferrin). Day 0 represents the day that the differentiation protocol is started, e.g. SFD media is introduced to the population of cells. This allows for potential waiting periods for even distribution of cells, plating of iPSCs, and the like. As such, the cells can be maintained in culture for a period of time prior to introduction of the SFD media. For example, cells may be maintained for up to seven days prior to day 0 introduction of SFD media. Without being bound by theory, this step of introduction of supplemented SFD medium induces hematopoietic and mesoderm differentiation. In some embodiments, the cells can be cultured in supplemented SFD medium for 3, 4, 5, 6, or 7 days. In some embodiments, the cells are cultured in supplemented SFD medium for 3 days. In some embodiments of the invention, BMP4 may be added to the SFD medium in a concentration range from 0.1-500 ng/ml, preferably 1-100 ng/ml, and even more preferably 5-25 ng/ml. In some embodiments, other BMPs or small molecules that activate ALK1, ALK2, and or ALK3 signaling can be added instead of or in addition to BMP4. In some embodiments, BMP2 or BMP8a may be added instead of or in addition to BMP4 in a concentration range of 1-200 ng/ml. In some embodiments, BMP4, other BMPs and/or small molecules that activate ALK1, ALK2, and or ALK3 signaling may be added to the media on day 0 to day 3. Without being bound by theory, BMP4 and other BMPs or small molecules that activate ALK1, ALK2, and or ALK3 signaling activates SMAD signaling to form mesoderm. In some embodiments, BMP4, other BMPs and/or small molecules that activate ALK1, ALK2, and or ALK3 signaling is a required component of this step of the invention. In some embodiments, bFGF may be added to the media in a concentration range from 1-500 ng/ml, preferably 10-100 ng/ml, and even more preferably 20-50 ng/ml. In some embodiments, other FGFs or MAPk agonists can be added instead of or in addition to bFGF. In some embodiments, bFGF, other FGFs and/or MAPk agonists may be added to the media on day 0 to day 3. Without being bound by theory, bFGF, other FGFs or MAPk agonists aid in survival and patterning to mesoderm. In some embodiments, bFGF, other FGFs and/or MAPk agonists is a required component of this step of the invention. In some embodiments, Y-27632 may be added to the media in a range from 100 nM-30 μM, preferably 1 μM-20 μM, and even more preferably 5 μM-20 μM. In some embodiments, Rho kinase inhibitors can be added instead of or in addition to Y-27632. In some embodiments, Y-27632 and/or Rho kinase inhibitors may be added to the media on day 0. Without being bound by theory, Y-27632 and/or Rho kinase inhibitors allow cells to survive as single cells for even distribution in the dish. In some embodiments, CHIR99021 may be added to the media in a range from 0.1-20 μM, preferably 1-10, and even more preferably 5-10 μM. In some embodiments, WNT proteins, other GSK3b inhibitors, and/or small molecules that lead to β-catenin stabilization, such as Wnt3a, FZM1.8, BIO lithium chloride, CHIR-98014, SB216763, SB415286 can be added instead of or in addition to CHIR99021. In some embodiments, Wnt3a may be added instead of or in addition to CHIR99021 in a concentration range of 1-200 ng/ml, FZM1.8 may be added instead of or in addition to CHIR99021 in a concentration range of 100 nM-100 μM, BIO may be added instead of or in addition to CHIR99021 in a concentration range of 100 nM-100 μM, lithium chloride may be added instead of or in addition to CHIR99021 in a concentration range of 0.1 mM-20 mM, CHIR-98014 may be added instead of or in addition to CHIR99021 in a concentration range of 500 nM-50 μM, SB216763 may be added instead of or in addition to CHIR99021 in a concentration range of 500 nM-50 μM, and/or SB415286 may be added instead of or in addition to CHIR99021 in a concentration range of 500 nM-50 μM. In some embodiments, CHIR99021, Wnt3a, FZM1.8, BIO lithium chloride, CHIR-98014, SB216763, and/or SB415286 may be added to the media on days 0 to 2, 1 to 2, or only on day 2. Without being bound by theory, CHIR99021, Wnt3a, FZM1.8, BIO lithium chloride, CHIR-98014, SB216763, and/or SB415286 activates Wnt signaling by inhibiting GSK3b. In some embodiments, CHIR99021, Wnt3a, FZM1.8, BIO lithium chloride, CHIR-98014, SB216763, and/or SB415286 is a required component of this step of the invention. In some embodiments, SB-431542 may be added to the media in a range from 0.1-20 μM. This was found to improve efficiency. In some embodiments, other means to inhibit SMAD signaling, including LY2109761, SB525334, SB505124, GW788388, LY364947, Galunisertib (LY2157299), and/or RepSox may be added instead of or in addition to SB-431542. In some embodiments, LY2109761 may be added instead of or in addition to SB-431542 in a concentration range of 500 nM-50 μM, SB525334 may be added instead of or in addition to SB-431542 in a concentration range of 500 nM-50 μM, SB505124 may be added instead of or in addition to SB-431542 in a concentration range of 500 nM-50 μM, GW788388 may be added instead of or in addition to SB-431542 in a concentration range of 500 nM-50 μM, LY364947 may be added instead of or in addition to SB-431542 in a concentration range of 500 nM-50 μM, Galunisertib (LY2157299) may be added instead of or in addition to SB-431542 in a concentration range of 500 nM-50 μM, and/or RepSox may be added instead of or in addition to SB-431542 in a concentration range of 500 nM-50 μM. In some embodiments, SB-431542, LY2109761, SB525334, SB505124, GW788388, LY364947, Galunisertib (LY2157299), and/or RepSox may be added to the media on days 1 to 3, on days 2 and 3, or only on day 3. Without being bound by theory, SB-431542, LY2109761, SB525334, SB505124, GW788388, LY364947, Galunisertib (LY2157299), and/or RepSox inhibit ALK/SMAD signaling. In some embodiments, the SFD media is supplemented with BMP4, bFGF, and CHIR99021, in the amounts and times described above. In some embodiments, the SFD media is supplemented with BMP4, bFGF, CHIR99021, and SB-431542, in the amounts and times described above. In one embodiment, the SFD media is supplemented with 10 μM Y-27632 on day 0; 10 ng/ml BMP4 on days 0, 1, and 2; 25 ng/ml bFGF on days 0, 1, and 2; 8 μM CHIR99021 on days 1 and 2; and 6 μM SB-431542 on day 2. The cells are cultured in this media for up to three days. This step is done under hypoxic conditions, which is at an O₂ concentration of less than 10%, preferably 5%, and a CO₂ concentration of between 1% and 10%, preferably 5%, at 32-39° C., preferably 37° C.

The next step is culturing the cells in StemPro-34 medium under hypoxic conditions, which is at an O₂ concentration of less than 10%, preferably 5%, and a CO₂ concentration of between 1% and 10%, preferably 5%, at 32-39° C., preferably 37° C. Without being bound by theory, this step induces endothelium formation. In some embodiments, the cells can be cultured in StemPro-34 medium under hypoxic conditions up to day 4, 5, 6, 7, 8, or 9. In some embodiments, the cells are cultured in supplemented SFD medium up to day 9. In some embodiments of the invention, bFGF may be added to the media in a range from 1-500 ng/ml, preferably 10-100 ng/ml, and even more preferably 20-50 ng/ml. In some embodiments, other FGFs or MAPk agonists can be added instead of or in addition to bFGF. In some embodiments, bFGF, other FGFs or MAPk agonists may be added to the media on day 3 up to day 14 or longer, such as up to day 15, 16, 17, 18, 19, 20, or 21. In some embodiments, SB-431542 may be added to the media in a range from 0.1-20 μM. In some embodiments, other means to inhibit SMAD signaling, including LY2109761, SB525334, SB505124, GW788388, LY364947, Galunisertib (LY2157299), and/or RepSox may be added instead of or in addition to SB-431542. In some embodiments, LY2109761 may be added instead of or in addition to SB-431542 in a concentration range of 500 nM-50 μM, SB525334 may be added instead of or in addition to SB-431542 in a concentration range of 500 nM-50 μM, SB505124 may be added instead of or in addition to SB-431542 in a concentration range of 500 nM-50 μM, GW788388 may be added instead of or in addition to SB-431542 in a concentration range of 500 nM-50 μM, LY364947 may be added instead of or in addition to SB-431542 in a concentration range of 500 nM-50 μM, Galunisertib (LY2157299) may be added instead of or in addition to SB-431542 in a concentration range of 500 nM-50 μM, and/or RepSox may be added instead of or in addition to SB-431542 in a concentration range of 500 nM-50 μM. In some embodiments, SB-431542, LY2109761, SB525334, SB505124, GW788388, LY364947, Galunisertib (LY2157299), and/or RepSox may be added to the media on day 3, or from day 3 to day 4 or longer, such as up to day 9. In some embodiments, VEGF may be added to the media in a range from 0.1-500 ng/ml, preferably 10-100 ng/ml, and even more preferably 20-50 ng/ml. In some embodiments, drugs that stimulate angiogenesis such as VEGF-C, angiopoietin-1, 2, 3, and/or 4, KDR/FLT-1 agonists, i/eNOS agonists and/or nitric oxide may be added instead of or in addition to VEGF. In some embodiments, VEGF-C, angiopoietin-1, 2, 3, and/or 4 may be added instead of or in addition to VEGF in a concentration range of 1-200 ng/ml. In some embodiments, VEGF may be added to the media on day 3 up to day 14 or longer, such as up to day 15, 16, 17, 18, 19, 20, or 21. Without being bound by theory, VEGF, VEGF-C, angiopoietin-1, 2, 3, and/or 4, KDR/FLT-1 agonists, i/eNOS agonists and/or nitric oxide promote endothelial cell formation and survival. In some embodiments, an HSC cocktail may be added to the media on day 6 up to day 21. The HSC cocktail can contain one or more of: SCF, IL-6, IL-3, FLT3L, IGF-1, IL-11, and EPO. In some embodiments, the HSC cocktail can contain one or more of: SCF, IL-6, IL-3, FLT3L, IGF-1, and/or IL-11, each in a concentration range of 1-200 ng/ml, and/or EPO in a concentration range of 0.1-20 U/ml. In one embodiment, the HSC cocktail contains 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO.

The next step is culturing the cells in StemPro-34 medium under non-hypoxic conditions, which is at an O₂ concentration of greater than 10% up to 30%, preferably normoxic levels or 15-20%, and a CO₂ concentration of between 1% and 10%, preferably 5%, 32-39° C., preferably 37° C. Without being bound by theory, this step induces endothelial-hematopoietic transition. In some embodiments, the cells are cultured in StemPro-34 medium under non-hypoxic conditions following StemPro-34 medium under hypoxic conditions (for example, from day 9) up to day 21 and beyond. In some embodiments, the cells can be cultured in StemPro-34 medium under non-hypoxic conditions up to day 5, 6, 7, 8, 9, 10, 11, 12, 13, 14, 15, 16, 17, 18, or 19. In some embodiments, the cells are cultured in StemPro-34 medium under non-hypoxic conditions up to day 14. In some embodiments of the invention, bFGF may be added to the media in a range from 1-500 ng/ml, preferably 5-50 ng/ml, and even more preferably 10-25 ng/ml. Other compounds that may be added instead of or in addition to bFGF are described above. In some embodiments, bFGF may be added to the media on day 3 up to day 14 or longer (administered both under hypoxic and non-hypoxic conditions), such as up to day 15, 16, 17, 18, 19, 20, or 21. In some embodiments, VEGF may be added to the media in a range from 0.1-500 ng/ml, preferably 10-100 ng/ml, and even more preferably 20-50 ng/ml. Other compounds that may be added instead of or in addition to VEGF are described above. In some embodiments, VEGF may be added to the media on day 3 up to day 14 or longer, such as up to day 15, 16, 17, 18, 19, 20, or 21. In some embodiments, an HSC cocktail may be added to the media on day 6 up to day 21. The HSC cocktail can contain one or more of: SCF, IL-6, IL-3, FLT3L, IGF-1, IL-11, and EPO. Ranges for HSC cocktail components are described above. In one embodiment, the HSC cocktail contains 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO. In some embodiments, EHT cocktail may be added to the media following StemPro-34 medium under hypoxic conditions (for example, from day 9) up to day 14 and beyond, and can be replaced each day. The EHT cocktail can contain one or more of: BMP4 in a concentration range of 1-200 ng/ml, SHH in a concentration range of 1-200 ng/ml, Angiotensin II in a concentration range of 0.1-100 μg/ml, and/or Losartan potassium in a concentration range of 1 μM-1000 μM. In some embodiments, SAG may be added instead of or in addition to SHH in a concentration range of 1-200 ng/ml, preferably 10 ng/ml. In one embodiment, the EHT cocktail contains 10 ng/ml BMP4, 10 ng/ml SHH, 10 ug/ml Angiotensin II, and 100 uM Losartan potassium, replaced each day.

The next step is culturing the cells in StemPro-34 medium under non-hypoxic expansion conditions, which is at an O₂ concentration of greater than 10% up to 30%, preferably normoxic levels or 15-20%, and a CO₂ concentration of between 1% and 10%, preferably 5%, 32-39° C., preferably 37° C. In some embodiments, the cells are cultured in StemPro-34 medium under non-hypoxic expansion conditions with HSC cocktail alone. Ranges for HSC cocktail components are described above. In some embodiments, the cells are cultured in StemPro-34 medium under non-hypoxic expansion conditions without EHT cocktail, VEGF, or bFGF. In some embodiments, the HSC cocktail is replaced every 3 days. The HSC cocktail can contain one or more of: SCF, IL-6, IL-3, FLT3L, IGF-1, IL-11, and EPO. In one embodiment, the HSC cocktail contains 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO. Following this step, HSCs are produced. In some embodiments, the HSCs express CXCR4 on the cell surface.

For example, the method of the present invention can include the following steps: (a) obtaining a population of pluripotent stem cells, (b) inducing hematopoietic differentiation by culturing on day 0 in SFD medium, 10 uM Y-27632, 10 ng/ml BMP4 and 25 ng/ml bFGF; culturing for 1-2 days with SFD medium, 10 ng/ml BMP4, 5 ng/ml bFGF, and 8 uM CHIR99021; culturing for 1 day with StemPro34 medium, 12.5 ng/ml bFGF, and 25 ng/ml VEGF; culturing for 1-2 day with StemPro34 medium, 12.5 ng/ml bFGF, and 25 ng/ml VEGF; culturing for 2-4 days with StemPro34 medium, 12.5 ng/ml bFGF, 25 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO; culturing for 3-5 days with StemPro34 medium, 12.5 ng/ml bFGF, 12.5 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, 2 U/ml EPO, 10 ng/ml BMP4, 10 ng/ml SHH, 10 ug/ml Angiotensin II, and 100 uM Losartan potassium, replaced each day; culturing for 5-10 days with StemPro34 medium, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO replaced every 3 days.

In one embodiment, the methods described above induce hematopoietic differentiation and generate lin−CD34+CD43+CD45+ cells. In some embodiments, hematopoietic cells and precursors of hematopoietic cells produced as part of the invention include cells expressing CXCR4, cells that are CD34+, CD45+, CD90+ and THY1+, cells that are CD38−, Lin−, CD43− or CD73−, cells that are CD45+, CD34+, CD90+, CD38−, and Lin−, cells expressing CD90, cells expressing runx1c, or any combination of the above. Runx1 is an essential gene for the onset of hematopoiesis, as deletion of RUNX1 causes embryonic lethality. It has also been suggested Runx1c isoform is more specifically expressed at the time of definitive hematopoiesis, while Runxla/b is expressed more broadly (Ng et al. (2016) Nat Biotechnol 34(11):1168-79; Challen et al. (2010) Exp Hematol 38(5):403-16; Sroczynska et al. (2009) Blood 114(26): 5279-89; Bos et al. (2015) Development 142(15):2719-24; Bee et al. (2010) Blood 115(15):3042-50).

One may also use the above identified invention to create cells of myeloid lineage from iPSCs. For example, one may obtain iPSCs as described in Yu et al. (2007) Science 318:1917-1920, and differentiate them into lin-CD34+CD43+CD45+ myeloid progenitors enriched cells. Starting from this point, one can then use the above-described protocol.

In some embodiments, the present invention provides definitive hematopoiesis and generation of long-term repopulating HSCs. In some embodiments, these long-term repopulating HSCs include cells expressing CXCR4, cells that are CD34+, CD45+, CD90+ and THY1+, cells that are CD38−, Lin−, CD43− or CD73−, cells that are CD45+, CD34+, CD90+, CD38−, and Lin−, cells expressing CD90, cells expressing runx1c, or any combination of the above. Without being bound by theory, the expression of CXCR4 is involved in homing of HSCs and long-term population of HSCs to the bone marrow. In some embodiments, HSCs of the present invention include HSCs generated using methods of the present invention, wherein the HSCs express CXCR4 on the cell surface.

The present invention has been described above with respect to its preferred embodiments. Other forms of this concept are also intended to be within the scope of the claims.

### Uses for Hematopoietic Cells and Precursors Thereof

The hematopoietic cells and hematopoietic precursor cells provided by methods and compositions of certain aspects of the invention can be used in a variety of applications. These include but are not limited to transplantation or implantation of the hematopoietic cells and hematopoietic precursor in vivo; screening cytotoxic compounds, carcinogens, mutagens growth/regulatory factors, pharmaceutical compounds, etc., in vitro; elucidating the mechanism of hematological diseases and injuries; studying the mechanism by which drugs and/or growth factors operate; diagnosing and monitoring cancer in a patient; gene therapy; and the production of biologically active products, to name but a few.

Programming-derived hematopoietic and hematopoietic precursor cells of this invention can be used to screen for factors (such as solvents, small molecule drugs, peptides, and polynucleotides) or environmental conditions (such as culture conditions or manipulation) that affect the characteristics of hematopoietic cells provided herein.

In some applications, stem cells (differentiated or undifferentiated) are used to screen factors that promote maturation of cells along the hematopoietic cell lineage, or promote proliferation and maintenance of such cells in long-term culture. For example, candidate hematopoietic cell maturation factors or growth factors are tested by adding them to stem cells in different wells, and then determining any phenotypic change that results, according to desirable criteria for further culture and use of the cells.

Particular screening applications of this invention relate to the testing of pharmaceutical compounds in drug research. The reader is referred generally to the standard textbook In vitro Methods in Pharmaceutical Research, Academic Press, 1997, and U.S. Pat. No. 5,030,015). In certain aspects of this invention, cells programmed to the hematopoietic lineage play the role of test cells for standard drug screening and toxicity assays, as have been previously performed on hematopoietic cells and precursors in short-term culture. Assessment of the activity of candidate pharmaceutical compounds generally involves combining the hematopoietic cells or precursors provided in certain aspects of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on hematopoietic cells or precursors, or because a compound designed to have effects elsewhere may have unintended effects on hematopoietic cells or precursors. Two or more drugs can be tested in combination (by combining with the cells either simultaneously or sequentially), to detect possible drug-drug interaction effects.

This invention also provides for the use of hematopoietic cells and hematopoietic precursor cells provided herein to restore a degree of function to a subject needing such therapy, perhaps due to a hematological disease or disorder or an injury. For example, hematopoietic cells and hematopoietic precursor cells derived by methods disclosed herein may be used to treat hematological diseases and disorders such as hemoglobinopathies, anemias, etc. In addition, hematopoietic cells and their precursors may be useful in supplying blood or blood cells (such as, for example, red blood cells, platelets, and neutrophil granulocytes) to subjects in need thereof (such as, for example, subjects in need of a blood transfusion or subjects having a hematological disorder). Such cells may be useful for the treatment of hematopoietic cell deficiencies caused by cell-suppressive therapies, such as chemotherapy.

To determine the suitability of hematopoietic cells and precursors provided herein for therapeutic applications, the cells can first be tested in a suitable animal model. At one level, cells are assessed for their ability to survive and maintain their phenotype in vivo. Programmed cells provided herein are administered to immunodeficient animals (such as NOG mice, or animals rendered immunodeficient chemically or by irradiation) at a site amenable for further observation, such as under the kidney capsule, into the spleen, into a liver lobule, or into the bone marrow. Tissues are harvested after a period of a few days to several weeks or more, and assessed as to whether starting cell types such as pluripotent stem cells are still present. This can be performed by providing the administered cells with a detectable label (such as green fluorescent protein, or β-galactosidase); or by measuring a constitutive marker specific for the administered human cells. Where programmed cells provided herein are being tested in a rodent model, the presence and phenotype of the administered cells can be assessed by immunohistochemistry or ELISA using human specific antibody, or by RT-PCR analysis using primers and hybridization conditions that cause amplification to be specific for human polynucleotide sequences. Suitable markers for assessing gene expression at the mRNA or protein level are provided elsewhere in this disclosure.

In some embodiments, the invention can be described as below:

Emb 1. A method of producing a hematopoietic precursor cell comprising the steps of:
a) obtaining a population of pluripotent stem cells;
b) culturing the cells on day 0 in supplemented serum-free differentiated (SFD) medium under a first hypoxic condition;
c) culturing the cells in StemPro-34 medium under a second hypoxic condition;
d) culturing the cells in StemPro-34 medium under non-hypoxic conditions; and
e) culturing the cells in StemPro-34 medium under non-hypoxic expansion conditions; and
f) collect population of hematopoietic precursor cells.

Emb 2. The method of Emb 1, wherein the pluripotent stem cells are human pluripotent stem cells.

Emb 3. The method of Emb 2, wherein the pluripotent stem cells are inducible pluripotent stem cells.

Emb 4. The method of Emb 2, wherein the pluripotent stem cells are embryonic stem cells.

Emb 5. The method of Emb 1, wherein the supplemented SFD medium is supplemented with one or more of: BMP4, bFGF, Y-27632, CHIR99021, and SB-431542 added to the SFD medium.

Emb 6. The method of Emb 5, wherein the BMP4 is at a range from 0.1-500 ng/ml.

Emb 7. The method of Emb 5, wherein the BMP4 is added to the medium on days 0, 1, or 2.

Emb 8. The method of Emb 5, wherein the BMP4 is added to the medium on days 0, 1, and 2.

Emb 9. The method of Emb 5, wherein the bFGF is at a range from 1-500 ng/ml.

Emb 10. The method of Emb 5, wherein the bFGF is added to the medium on days 0, 1, or 2.

Emb 11. The method of Emb 5, wherein the bFGF is added to the medium on days 0, 1, and 2.

Emb 12. The method of Emb 5, wherein the Y-27632 is at a range from 100 nM-30 μM.

Emb 13. The method of Emb 5, wherein the Y-27632 is added to the medium on day 0.

Emb 14. The method of Emb 5, wherein the CHIR99021 is at a range from 0.1-20 μM.

Emb 15. The method of Emb 5, wherein the CHIR99021 is added to the medium on days 0, 1, or 2.

Emb 16. The method of Emb 5, wherein the CHIR99021 is added to the medium on days 1 and 2.

Emb 17. The method of Emb 5, wherein the SB-431542 is at a range from 0.1-20 μM.

Emb 18. The method of Emb 5, wherein the SB-431542 is added to the medium on day 0, 1, or 2.

Emb 19. The method of Emb 5, wherein the SB-431542 is added to the medium on day 2.

Emb 20. The method of Emb 5, wherein the BMP4, bFGF, Y-27632, CHIR99021, and SB-431542 are added to the medium.

Emb 21. The method of Emb 20, wherein the BMP4 is at a concentration range of 5-25 ng/ml and added to the medium on days 0, 1, and 2; the bFGF is at a concentration range of 20-50 ng/ml and added to the medium on days 0, 1, and 2; the Y-27632 is at a concentration range of 5 μM-20 μM and added to the medium on day 0; the CHIR99021 is at a concentration range of 5 μM-20 μM and added to the medium on days 1 and 2; and the SB-431542 is at a concentration range of 0.1-20 μM and added to the medium on day 2.

Emb 22. The method of Emb 21, wherein the BMP4 is at a concentration of 10 ng/ml and added to the medium on days 0, 1, and 2; the bFGF is at a concentration of 25 ng/ml and added to the medium on days 0, 1, and 2; the Y-27632 is at a concentration of 10 μM and added to the medium on day 0; the CHIR99021 is at a concentration range of 5 μM-20 μM and added to the medium on days 1 and 2; and the SB-431542 is at a concentration range of 0.1-20 μM and added to the medium on day 2.

Emb 23. The method of Emb 1, wherein the StemPro-34 medium under a second hypoxic condition is supplemented with one or more of: bFGF, HSC cocktail, SB-431542, and VEGF added to the StemPro-34 medium under a second hypoxic condition.

Emb 24. The method of Emb 23, wherein the bFGF is at a range from 20-50 ng/ml.

Emb 25. The method of Emb 23, wherein the bFGF is added to the medium on days day 3 up to day 14.

Emb 26. The method of Emb 23, wherein the HSC cocktail comprises one or more of: SCF, IL-6, IL-3, FLT3L, IGF-1, IL-11, and EPO.

Emb 27. The method of Emb 23, wherein the HSC cocktail comprises SCF, IL-6, IL-3, FLT3L, IGF-1, and/or IL-11, each in a concentration range of 1-200 ng/ml, and/or EPO in a concentration range of 0.1-20 U/ml.

Emb 28. The method of Emb 23, wherein the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO.

Emb 29. The method of Emb 23, wherein the HSC cocktail is added to the medium on day 6 up to day 21.

Emb 30. The method of Emb 23, wherein the SB-431542 is at a range from 0.1-20 μM.

Emb 31. The method of Emb 23, wherein the SB-431542 is added to the medium on day 3 to day 9.

Emb 32. The method of Emb 23, wherein the VEGF is at a range from 20-50 ng/ml.

Emb 33. The method of Emb 23, wherein the VEGF is added to the medium on day 3 to day 14.

Emb 34. The method of Emb 23, wherein the bFGF, HSC cocktail, SB-431542, and VEGF are added to the medium.

Emb 35. The method of Emb 34, wherein the bFGF is at a concentration range from 20-50 ng/ml and is added to the medium on day 3 up to day 14; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 up to day 21; the SB-431542 is at a concentration range from 0.1-20 μM and is added to the medium on day 3 to day 9; and the VEGF is at a concentration range from 20-50 ng/ml and is added to the medium on day 3 to day 14.

Emb 36. The method of Emb 34, wherein the bFGF is at a concentration of 12.5 ng/ml and is added to the medium on day 3 to day 9; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 to day 9; the SB-431542 is at a concentration of 6 μM and is added to the medium on day 3; and the VEGF is at a concentration of 25 ng/ml and is added to the medium on day 3 to day 9.

Emb 37. The method of Emb 1, wherein the StemPro-34 medium under non-hypoxic condition is supplemented with one or more of: bFGF, HSC cocktail, VEGF, and EHT cocktail added to the StemPro-34 medium under non-hypoxic condition.

Emb 38. The method of Emb 37, wherein the bFGF is at a range from 10-25 ng/ml.

Emb 39. The method of Emb 37, wherein the bFGF is added to the medium on day 3 to day 14.

Emb 40. The method of Emb 37, wherein the bFGF is added to the medium on day 9 to day 14.

Emb 41. The method of Emb 37, wherein the HSC cocktail comprises at least one of SCF, IL-6, IL-3, FLT3L, IGF-1, IL-11, and EPO.

Emb 42. The method of Emb 37, wherein the HSC cocktail comprises SCF, IL-6, IL-3, FLT3L, IGF-1, and/or IL-11, each in a concentration range of 1-200 ng/ml, and/or EPO in a concentration range of 0.1-20 U/ml.

Emb 43. The method of Emb 37, wherein the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO.

Emb 44. The method of Emb 37, wherein the HSC cocktail is added to the medium on day 6 to day 21.

Emb 45. The method of Emb 37, wherein the VEGF is at a range from 20-50 ng/ml.

Emb 46. The method of Emb 37, wherein the VEGF is added to the medium on day 3 to day 14.

Emb 47. The method of Emb 37, wherein the EHT cocktail comprises at least one of BMP4, SHH, Angiotensin II, and Losartan potassium.

Emb 48. The method of Emb 37, wherein the EHT cocktail comprises BMP4 in a concentration range of 1-200 ng/ml, SHH in a concentration range of 1-200 ng/ml, Angiotensin II in a concentration range of 0.1-100 μg/ml, and/or Losartan potassium in a concentration range of 1 μM-1000 μM.

Emb 49. The method of Emb 37, wherein the EHT cocktail is added to the medium on day 9 to day 14.

Emb 50. The method of Emb 23, wherein the bFGF, HSC cocktail, VEGF, and EHT cocktail are added to the medium.

Emb 51. The method of Emb 34, wherein the bFGF is at a concentration range from 10-25 ng/ml and is added to the medium on day 3 up to day 14; the HSC cocktail comprises SCF, IL-6, IL-3, FLT3L, IGF-1, and/or IL-11, each in a concentration range of 1-200 ng/ml, and/or EPO in a concentration range of 0.1-20 U/ml and is added to the medium on day 6 up to day 21; the VEGF is at a concentration range from 20-50 ng/ml and is added to the medium on day 3 to day 14; and the EHT cocktail comprises BMP4, SHH, Angiotensin II, and Losartan potassium and is added to the medium on day 9 to day 14.

Emb 52. The method of Emb 34, wherein the bFGF is at a concentration of 12.5 ng/ml and is added to the medium on day 9 to day 14; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 up to day 21; the VEGF is at a concentration of 12.5 ng/ml and is added to the medium on day 9 to day 14; and the EHT cocktail comprises BMP4, SHH, Angiotensin II, and Losartan potassium and is added to the medium on day 9 to day 14.

Emb 53. The method of Emb 1, wherein the StemPro-34 medium under non-hypoxic expansion condition is supplemented with HSC cocktail added to the StemPro-34 medium under non-hypoxic expansion condition.

Emb 54. The method of Emb 53, wherein the HSC cocktail comprises at least one of SCF, IL-6, IL-3, FLT3L, IGF-1, IL-11, and EPO.

Emb 55. The method of Emb 53, wherein the HSC cocktail comprises SCF, IL-6, IL-3, FLT3L, IGF-1, and/or IL-11, each in a concentration range of 1-200 ng/ml, and/or EPO in a concentration range of 0.1-20 U/ml.

Emb 56. The method of Emb 53, wherein the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO.

Emb 57. The method of Emb 53, wherein the HSC cocktail is added to the medium on day 6 to day 21.

Emb 58. The method of Emb 1, wherein the first hypoxic condition contains an 02 concentration less than 10%.

Emb 59. The method of Emb 1, wherein the second hypoxic condition contains an O₂ concentration less than 10%.

Emb 60. The method of Emb 1, wherein the step of culturing the cells in StemPro-34 medium under non-hypoxic conditions contains an O₂ concentration greater than 10%.

Emb 61. The method of Emb 1, wherein the step of culturing the cells in StemPro-34 medium under non-hypoxic expansion conditions contains an O₂ concentration greater than 10%.

Emb 62. A method of producing a hematopoietic precursor cell from a pluripotent stem cell or transdifferentiation of a somatic cell, comprising culturing the pluripotent stem cell or somatic cell under conditions to generate the hematopoietic precursor cell that can differentiate into different hematopoietic lineage cells, comprising the steps of (a) obtaining a population of pluripotent stem cells, (b) inducing hematopoietic differentiation by culturing on day 0 in SFD medium, 10 uM Y-27632, 10 ng/ml BMP4 and 25 ng/ml bFGF; culturing for 1-2 days with SFD medium, 10 ng/ml BMP4, 5 ng/ml bFGF, and 8 uM CHIR99021; culturing for 1 day with StemPro34 medium, 12.5 ng/ml bFGF, and 25 ng/ml VEGF; culturing for 1-2 day with StemPro34 medium, 12.5 ng/ml bFGF, and 25 ng/ml VEGF; culturing for 2-4 days with StemPro34 medium, 12.5 ng/ml bFGF, 25 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO; culturing for 3-5 days with StemPro34 medium, 12.5 ng/ml bFGF, 12.5 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, 2 U/ml EPO, 10 ng/ml BMP4, 10 ng/ml SHH, 10 ug/ml Angiotensin II, and 100 uM Losartan potassium, replaced each day; culturing for 5-10 days with StemPro34 medium, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO replaced every 3 days.

Emb 63. The method of Emb 62, wherein the media with StemPro34 medium, 12.5 ng/ml bFGF, and 25 ng/ml VEGF further comprises 6 uM SB 431542.

Emb 64. The method of Emb 62 or 63, wherein the media on days 2, 3, 4, or 5 further comprise 6 μm SB 431542 (TOCRIS).

Emb 65. The method of any one of Embs 1-64, wherein the pluripotent stem cell is an induced pluripotent stem cell.

Emb 66. The method of any one of Embs 1-64, wherein the pluripotent stem cell is an embryonic stem cell.

Emb 67. The method of any one of Embs 1-66, wherein the pluripotent stem cell is capable of homing to bone marrow.

Emb 68. The method of Emb 67, wherein the hematopoietic precursor cell expresses CXCR4.

Emb 69. The method of Emb 68, wherein the hematopoietic precursor cell expresses CXCR4 on the cell surface.

Emb 70. The method of any one of Embs 1-69, wherein the hematopoietic precursor cell is CD34+, CD45+, CD90+, or THY1+.

Emb 71. The method of Emb 70, wherein the hematopoietic precursor cell is CD34+, CD45+, CD90+ and THY1+.

Emb 72. The method of any one of Embs 1-71, wherein the hematopoietic precursor cell is CD38−, Lin−, CD43− or CD73−.

Emb 73. The method of Emb 72, wherein the hematopoietic precursor cell is CD38−, Lin−, CD43−, and CD73−.

Emb 74. The method of any one of Embs 1-73, wherein the hematopoietic precursor cell is CD45+, CD34+, CD90+, CD38−, and Lin−.

Emb 75. The method of any one of Embs 1-74, wherein the hematopoietic precursor cell is CD90+.

Emb. 76. The method of any one of Embs 1-75, wherein the hematopoietic precursor cell expresses runx1c.

Emb 77. A hematopoietic precursor cell produced using any of the methods of Embs 1-76.

Emb 78. The hematopoietic precursor cell of Emb 77, wherein said cell is capable of long term bone marrow engraftment.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

The following examples, including the experiments conducted and results achieved, are provided for illustrative purposes only and are not to be construed as limiting the present invention.

### EXAMPLES

### Example 1: Process for Generation of Hematopoietic Stem Cells

iPSC was added in a 6-well plate, coated with poly-L-Ornithine (PLO; Sigma) at a 1:7 dilution in PBS, with lml in each well and incubated at 37° C. for 2 hours. The PLO solution was replaced with Laminin (Sigma) in DMEM/F12 at 1:150 dilution, with lml in each well and incubated at 37° C. for 2 hours.

On day 0, the iPSC were lifted using TrypLE (Thermo Fisher) and 600,000 cells were seeded per well in 2 ml SFD medium (75:25 of IMDM:Ham's F-12, 0.05% BSA, 1×B27, 0.5×N2 supplements, 1× GlutaMax and 1× Penicillin-Streptomycin, 0.5 mM ascorbic acid, 450 pM Monothioglycerol, and 150 μg/mL holo-transferrin (R&D Systems))+10 uM Y-27632+10 ng/ml BMP4+25 ng/ml bFGF.

On day 1, the media was replaced with SFD medium+10 ng/ml BMP4+25 ng/ml bFGF+8 uM CHIR99021, adding 2 ml in each well. On days 2, 3, 4, and 5, 6 μM SB-431542 (TOCRIS) was added into the media in some samples. On day 3, the media was replaced with StemPro34 medium+12.5 ng/ml bFGF+25 ng/ml VEGF+6 uM SB 431542, adding 2 ml in each well and was incubated for 24 hours. On day 4, the media was replaced with StemPro34 medium+12.5 ng/ml bFGF+25 ng/ml VEGF, adding 2 ml in each well and was incubated for 48 hours. On days 6-8, the media was replaced with StemPro34 medium+12.5 ng/ml bFGF+25 ng/ml VEGF+50 ng/ml SCF+25 ng/ml IL-6+25 ng/ml IL-3+25 ng/ml FLT3L+25 ng/ml IGF-1+5 ng/ml IL-11+2 U/ml EPO, adding 2 ml in each well. On days 9-13, the media was replaced with StemPro34 medium+12.5 ng/ml bFGF+12.5 ng/ml VEGF+50 ng/ml SCF+25 ng/ml IL-6+25 ng/ml IL-3+25 ng/ml FLT3L+25 ng/ml IGF-1+5 ng/ml IL-11+2 U/ml EPO+10 ng/ml BMP4+10 ng/ml SHH+10 ug/ml Angiotensin II+100 uM Losartan potassium, adding 2 ml in each well, with the media replaced every 2-3 days. On days 14-21, the media was replaced with StemPro34 medium+50 ng/ml SCF+25 ng/ml IL-6+25 ng/ml IL-3+25 ng/ml FLT3L+25 ng/ml IGF-1+5 ng/ml IL-11+2 U/ml EPO, adding 2 ml in each well, with the media replaced every 3 days. On day 21, the cells were FACS sorted for Lin−(CD45RA, CD10, CD7, CD3, CD19, CD33, CD66b)CD34+CD45+CD38−CD90+ cells. On days 0-10, the cells were incubated at 37° C., 5% 02 and 5% CO₂. On days 11-21, the cells were incubated at 37° C., 20% 02 and 5% C02.

### Example 2: Assay for Presence of Hemogenic Endothelium and Hematopoietic Stem Cells

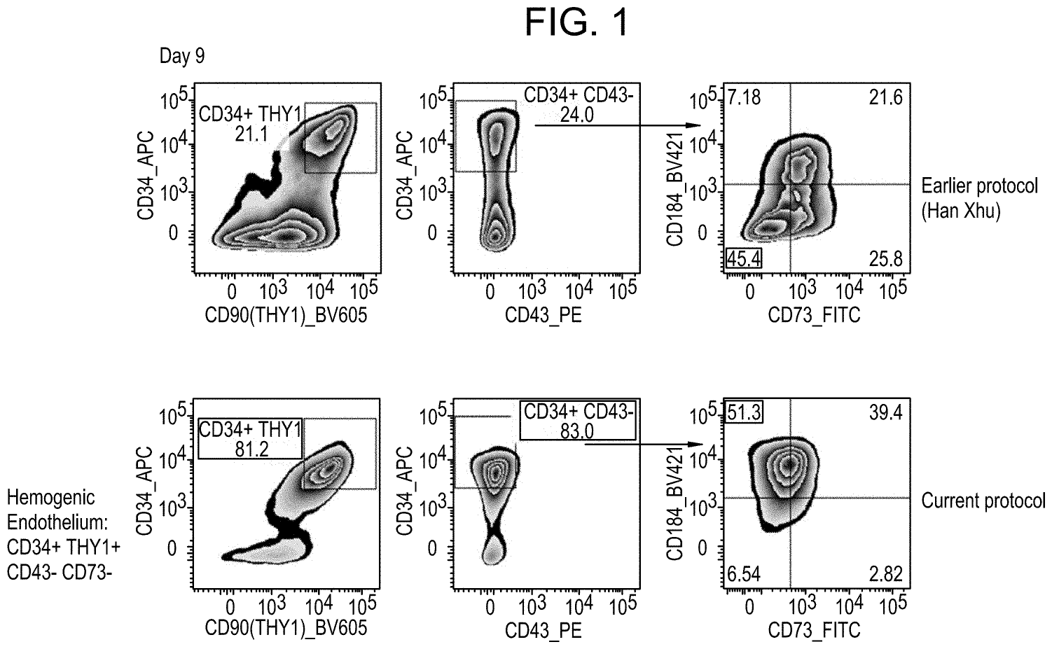

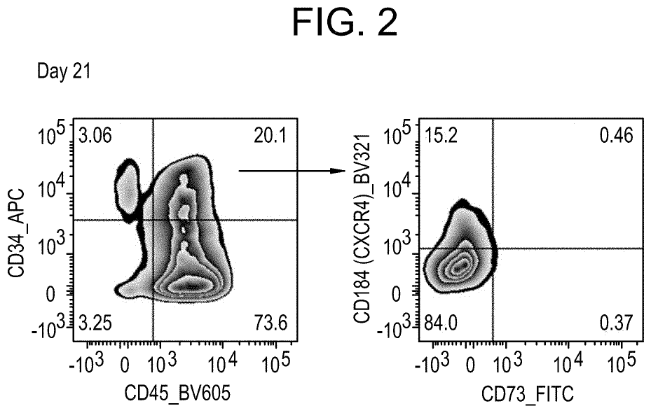

Using the protocol described in Example 1, cells from culture day 9 and 10 were sequenced using single cell sequencing. Hemogenic endothelium are a subset of endothelial cells capable of differentiating into hematopoietic cells. Hemogenic endothelium are characterized as CD34+THY1+CD43−CD73−. FACS plots showing the presence of hemogenic endothelium are shown in FIG. 1, both in an earlier protocol, as well as the current protocol shown in Example 1. Further analysis for hematopoietic stem cells (CD34+CD45+CD73−) showed a window of endothelial to hematopoietic transition in iPSC cultures to days 19-21 of differentiation. These results are shown in FIG. 2.

### Example 3: Limiting Dilution Assay to Measure Multi-Lineage Potential of iPSC-Derived HSC

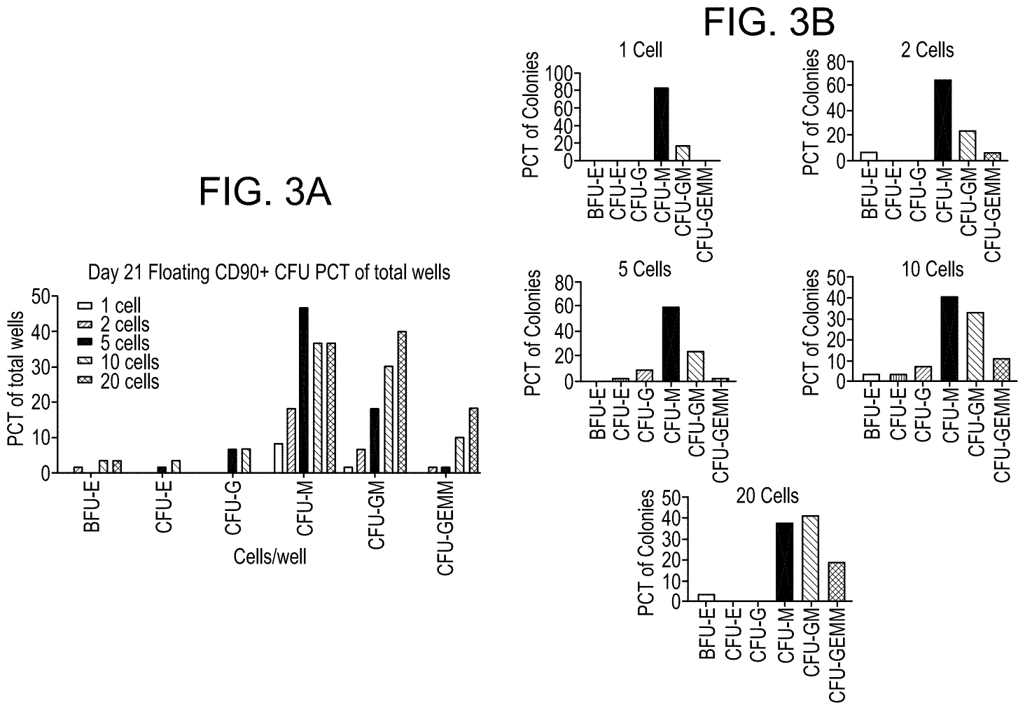

FACS was used to purify iPSC-derived putative HSCs (CD34+CD45+CD90+CD38−Lin−). Cells were loaded into wells at 20, 10, 5, 2, or 1 cell(s)/well, each well loaded with methylcellulose with permissive cytokines. The cells were cultured for 14 days and the colonies were scored for colony forming units. The results are shown in FIG. 3. FIG. 3A shows the percent of the wells having each cell type when the wells were loaded with a different number of cells. Depending on the number of cells loaded per well, different fractions of cells, including erythroid burst-forming units (BFU-E), macrophage CFU (CFU-M), granulocyte-macrophage CFU (CFU-GM), eosinophil colony-forming units (CFU-E), granulocyte CFU (CFU-G), and multipotential CFU (CFU-GEMM) formed colonies, as shown in FIG. 3B.

### Example 4: Generation of Runx1C-GFP Genetic Reporter System

Runx1 is an essential gene for the onset of hematopoiesis, as deletion of RUNX1 causes embryonic lethality. It has also been suggested Runx1c isoform is more specifically expressed at the time of definitive hematopoiesis, while Runx1a/b is expressed more broadly (Ng et al. (2016) Nat Biotechnol 34(11):1168-79; Challen et al. (2010) Exp Hematol 38(5):403-16; Sroczynska et al. (2009) Blood 114(26): 5279-89; Bos et al. (2015) Development 142(15):2719-24; Bee et al. (2010) Blood 115(15):3042-50).

The purpose of creating a GFP-2A-Runx1c genetic reporter line is to fluorescently label the nascent hematopoietic stem cells (HSC) emerging from hemogenic endothelium to allow for expression analysis of runx1c. This reporter line enabled us to visually determine the efficiency of our HSC differentiation protocol, and provided a straitforward readout.

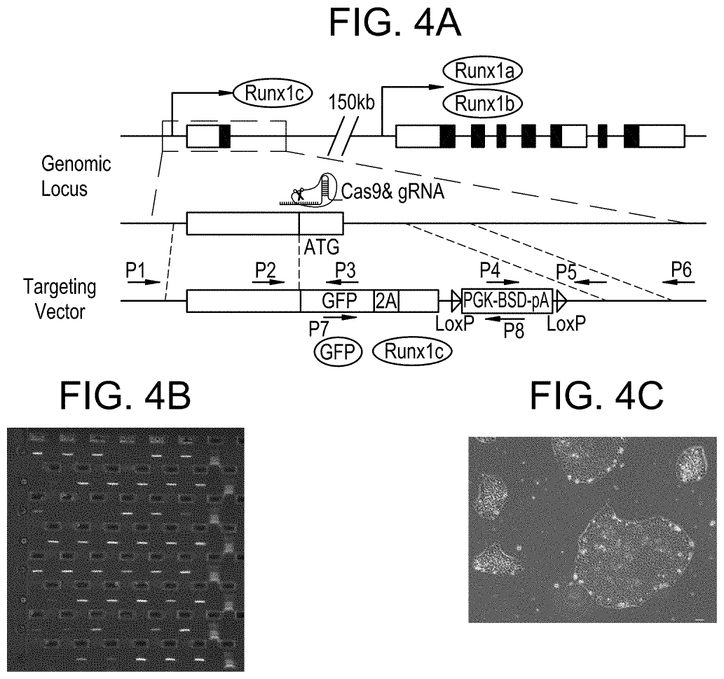

The targeting design and vector were constructed by using the following steps. Runx1c N-terminus targeting guide RNA 5′-GCATTTTCAGGAGGAAGCGA-3′ (SEQ ID NO:1) was cloned into pCas9-Guide vector (ORIGENE) using BamHI/BsmBI. The generation of GFP-2A-Runx1c hiPSC reporter line for labeling of hematopoietic stem cells (HSCs) is shown in FIG. 4. The “GFP-2A” sequence was inserted before the ATG start codon of Runx1c exon1, a “LoxP-PGK-BSD-pA-LoxP” cassette was also inserted in intron 1 for enrichment of correctly targeted human induced pluripotent stem cells (hiPSC) clones. The homology arm flanking the knock-in sequence consists of 1 kb upstream and downstream of guide RNA targeting site. 7.5 ug pCas9-Ruxn1c-Guide vector and 7.5 ug of GFP-2A-Runx1c donor vector were transfected into 2×10⁶ iPSCs using Lipofectamine 3000. 48 hours post transfection, 2.5 ug/ml blasticidin was applied to enrich targeted population. Cells were selected for 5-7 days and expanded for cryopreservation. FIG. 4A shows a schematic picture showing the strategy to target Runx1c genomic locus. Meanwhile, 1×10⁶ blasticidin-enriched iPSCs were harvested for genomic DNA isolation and PCR genotyping test. FIG. 4B shows that the primers described in 4A was used for screening positive colonies after genome editing. After blasticidin selection, a total of 48 single cell clones were picked, expanded and subjected PCR genotyping analysis. 38 clones exhibited positive genotyping band on agarose gel (efficiency=79%). FIG. 4C shows an image of the selected positive clone of GFP-2A-Runx1c hiPSC line.

The following primers in Table 1 were used for genotyping and sequencing of different regions of targeted Runx1c locus:

TABLE 1
Primers used for genotyping

| | | | |
|---|---|---|---|
| P1 | LH- | 5′-CTGAAAGAGATACATACTAA | Use P1/P3 for |
| | Out-F | AGTTGTCC (SEQ ID NO: 2) | amplifying |
| | | | left arm |
| P2 | LH-In- | 5′- AGTCCCAGAGGTATCCAGC | Use P2/P3 for |
| | F | AGAGG (SEQ ID NO: 3) | amplifying |
| | | | left junction |
| P3 | GFP-R | 5′- GTAGTTGCCGTCGTCCTTG | |
| | | AAGAAG (SEQ ID NO: 4) | |
| P4 | BSD-F | 5′- GCCATAGTGAAGGACAGTG | |
| | | ATGGAC (SEQ ID NO: 5) | |
| P5 | RH-In- | 5′- TCACAAACAAGACAGGGAA | Use P4/P5 for |
| | R | CTGGCA (SEQ ID NO: 6) | amplying |
| | | | right junction |
| P6 | RH- | 5′- CAGATACAATTTGGGTGCT | Use P4/P6 for |
| | Out-R | CAAGAGAG (SEQ ID NO: 7) | amplifying |
| | | | right arm |
| P7 | GFP-F | 5′- CTTCTTCAAGGACGACGGC | Use P7/P8 for |
| | | AACTAC (SEQ ID NO: 8) | amplying |
| | | | knock-in |
| | | | region |
| P8 | BSD-R | 5′- GTCCATCACTGTCCTTCAC | |
| | | TATGGC (SEQ ID NO: 9) | |

PCR was performed using PfuUltra II Hotstart PCR Master Mix (Agilent), using 100 ng genomic DNA of enriched transfection pool. Purified PCR product sequences were confirmed by Sanger sequencing (Genewiz).

For single cell cloning, blasticidin resistant iPSCs were dissociated into single cells by TryPLE and seeded at single cell density (˜2500 cells per 10-cm dish) in mTeSR media. CloneR (Stem Cell Technologies) was added for the first 4 days to promote survival and growth of single cell clones. A second round of blasticidin selection was applied from day 4-7 to further enrich positively targeted clones. Around Day 8-10, colonies emerged from single cell were picked under a microscope in tissue culture cabinet and transferred to 96-well Matrigel coated plates for continuing culture.

For passaging colony plates, when colonies in the 96-well plate grew to near confluent, cells were dissociated using ReLeSR (Stem Cell Technologies) and resuspended in mTeSR supplemented with 10 uM Y-27632 (TOCRIS). The cell suspension were then split into 3×96-well replicate plates at ratio of 1:3, 1:5 and 1:8, respectively. The 1:5 plate was again dissociated for cryopreservation a few days later.

To perform PCR screening, when the cells in the 1:3 plate grew to full confluent, they were lysed using 50 ul/well QuickExtract™ DNA Extraction Solution (Lucigen) according to manufacturer's instructions. 3 ul of DNA extraction solution was used as PCR template with primer set LH-In-F/GFP-R for PCR screening. Selected PCR positive colonies were confirmed by PCR with additional primer sets listed in Step 3 and Sanger sequencing.

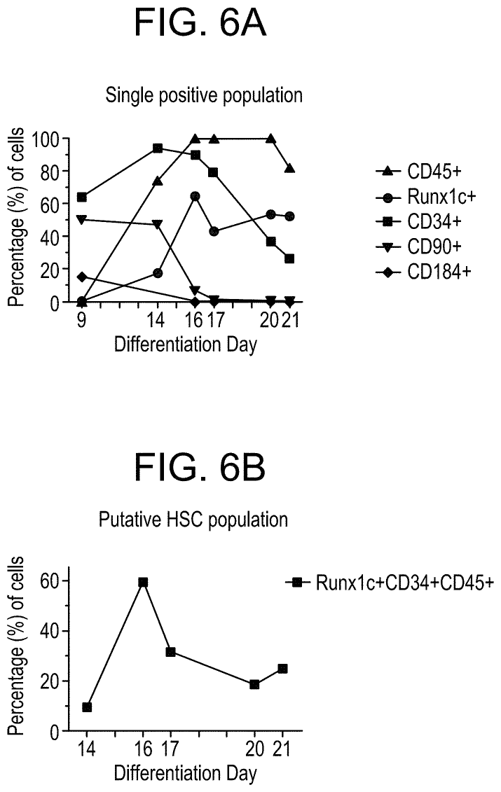

Confirmed GFP-2A-Runx1c hiPSC clones were expanded from the 1:8 replicate plate for downstream applications. Our data showed that RUNX1C-GFP temporal expression highly overlaps with existing HSC markers CD34 and CD45, but only marks a subpopulation of CD34/CD45 double positive population (see FIG. 6). Runx1c-GFP thus serves as an additional marker to further refine the HSC population for higher purity and efficacy.

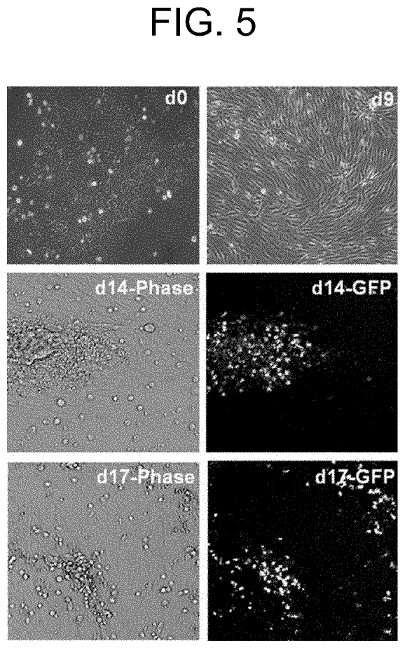

FIG. 5 shows a visualization of GFP positive hematopoietic stem cells in hiPSC differentiation: GFP-2A-Runx1c iPSCs (d0, top left panel) were firstly differentiated into endothelium (d9, top right panel), followed by induction of endothelial-hematopoietic transition (EHT) that results in emergence of GFP positive hematopoietic stem cells (d14, mid panel) from selected regions (dashed box, “blood island”) of GFP negative endothelial layer. At day 17, the production of GFP positive HSCs are no longer restricted in certain regions, but became more prominent throughout the tissue culture (d17 bottom panel).

FIG. 6 shows a time course of surface marker expression pattern of GFP-2A-Runx1c iPSCs during hematopoietic differentiation: (A) Single positive population. (B) Runx1c+CD34+CD45+ putative hematopoietic stem cell population.

### Example 5: Long-Term iPSC Cell Marker Expression Assay

CD34 and GFP-Runx1c Expression Over Time

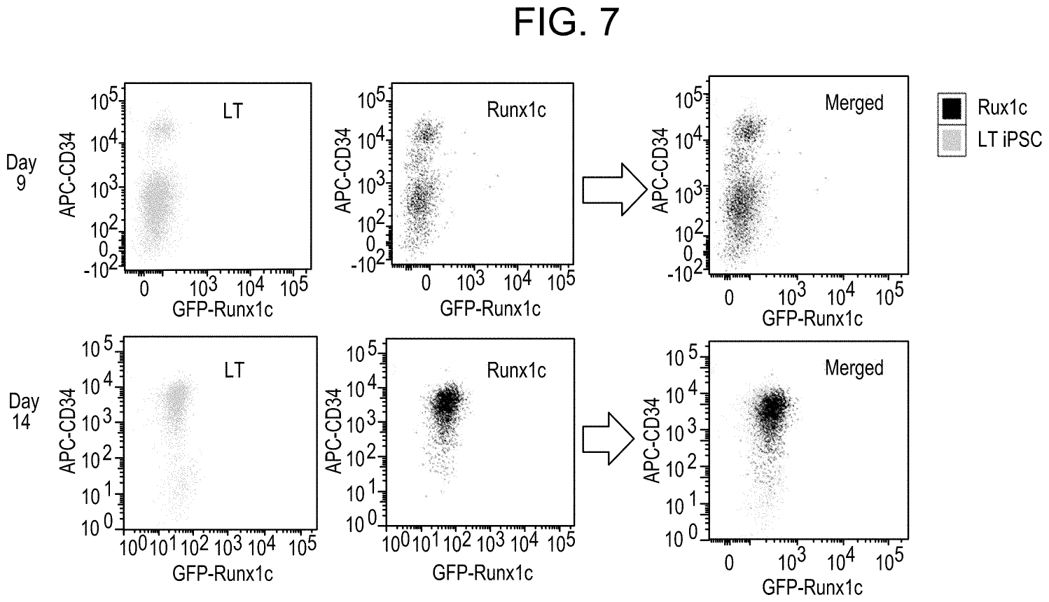

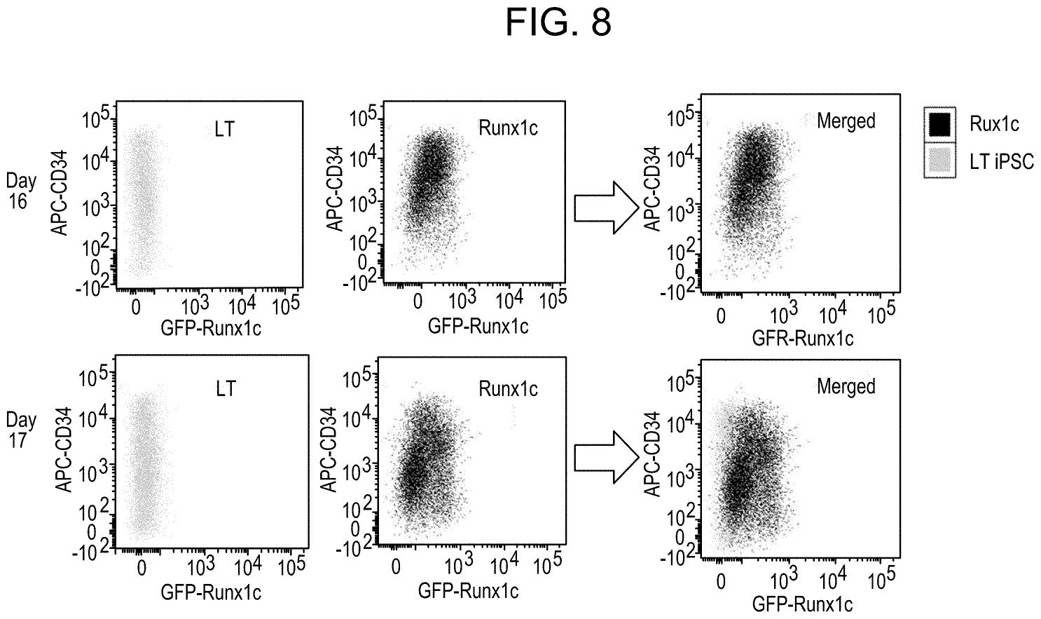

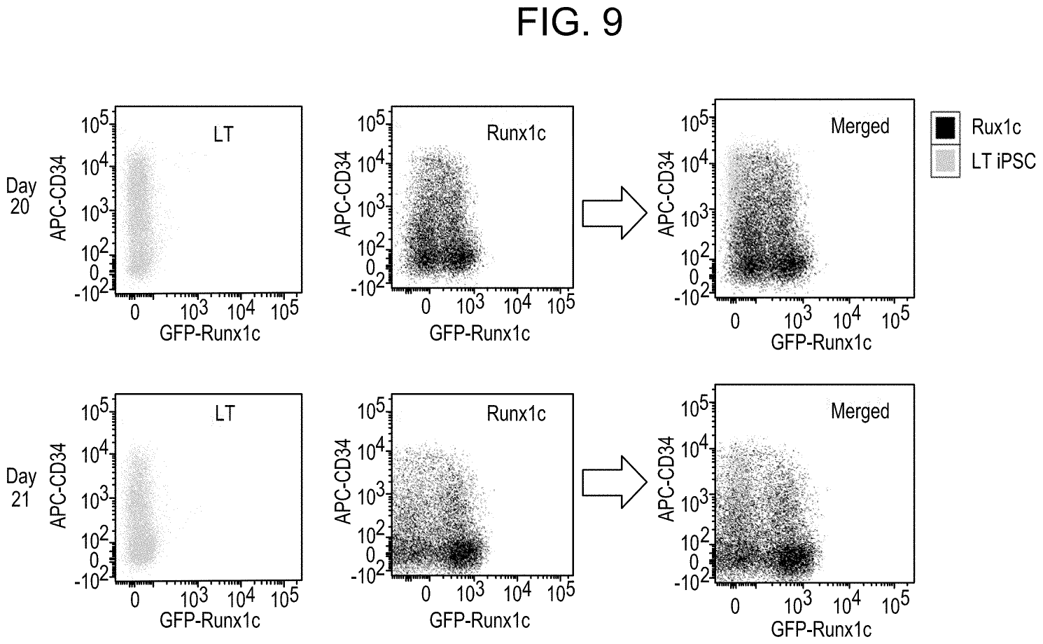

LT-iPSC and GFP-Runx1c stably expressed LT-iPSC were differentiated using the protocol of Example 1. Attached cells of D9 and suspension cells from Day 14, 16, 17, 20 and 21 were harvested for FACS analysis. All sample groups for FACS were stained with APC-CD34 and sytox blue (Thermo Fisher). FACS analysis were gated on single cells with negative sytox blue staining. FIG. 7 shows HSC CD34 vs GFP-Runx1c expression on days 9 and 14. FIG. 8 shows HSC CD34 vs GFP-Runx1c expression on days 16 and 17. FIG. 9 shows HSC CD34 vs GFP-Runx1c expression on days 20 and 21. In each figure, from left to right were LT-iPSC, GFP-Runx1c over-expressed iPSC and overlay of both cells. GFP-Runx1c started to show expression on Day 14, and expression increased over the time. From Day 14-17, all GFP-Runx1c positive cells were CD34+. Starting from day 20, GFP-Runx1c cells shifted to CD34-.

Different Cell Population Expression Over Time

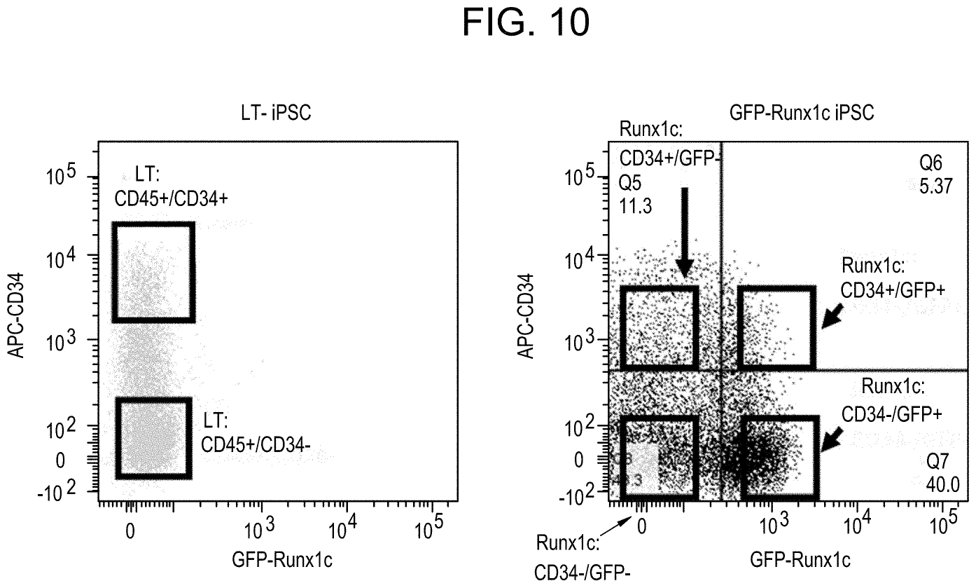

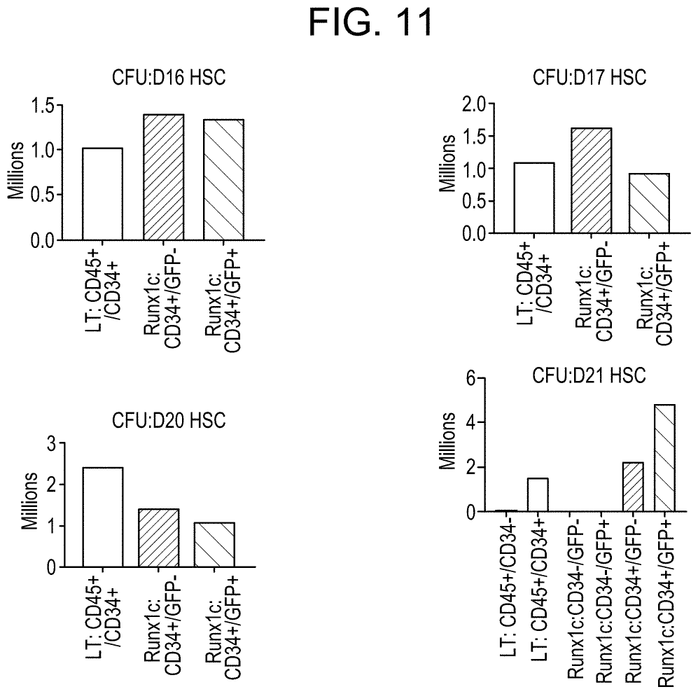

Different HSC populations were purified by flow cytometry (FACS) sorting. 5000 HSCs from each population were cultured in 5 ml MethoCult™ H4435 Enriched (from STEMCELL Technologies Inc.) in 6-well plates (37° C. with 5% CO₂). After 21 days of culture, all cells in MethoCult™ were collected and diluted in DMEM/F12. After spin down at 1000g×5 min, cell pellets were repeatedly titrated by P1000 pipette and single cell numbers were counted by ViaCell. HSC from LT-iPSC and GFP-Runx1c iPSC were sorted based on the gate strategy described above. On Day 16, 17 and 20, only LT: CD45+/CD34+ were sorted from LT-iPSC and Runx1c: CD34+/GFP− and Runx1c: CD34+/GFP− were sorted. On Day 21, all six populations were sorted for CFU assays, as shown in FIG. 10. FIG. 10 shows cell population sorting for CFU assays from LT-iPSC and GFP-Runx1c iPSC. All HSC from Runx1c-GFP were all gated on CD45+ cells first. All populations represent CD45+ cells. At early stages, GFP-Runx1c expressed HSC generated similar or lower total CFU cells; however, on Day 21, HSC GFP-Runx1c and CD34+ double positive HSCs are more robust in generating more cells from CFUs (as shown in FIG. 11). In all groups, CD34+ is critical to maintain CFU potential.

Cell Type Marker Analysis

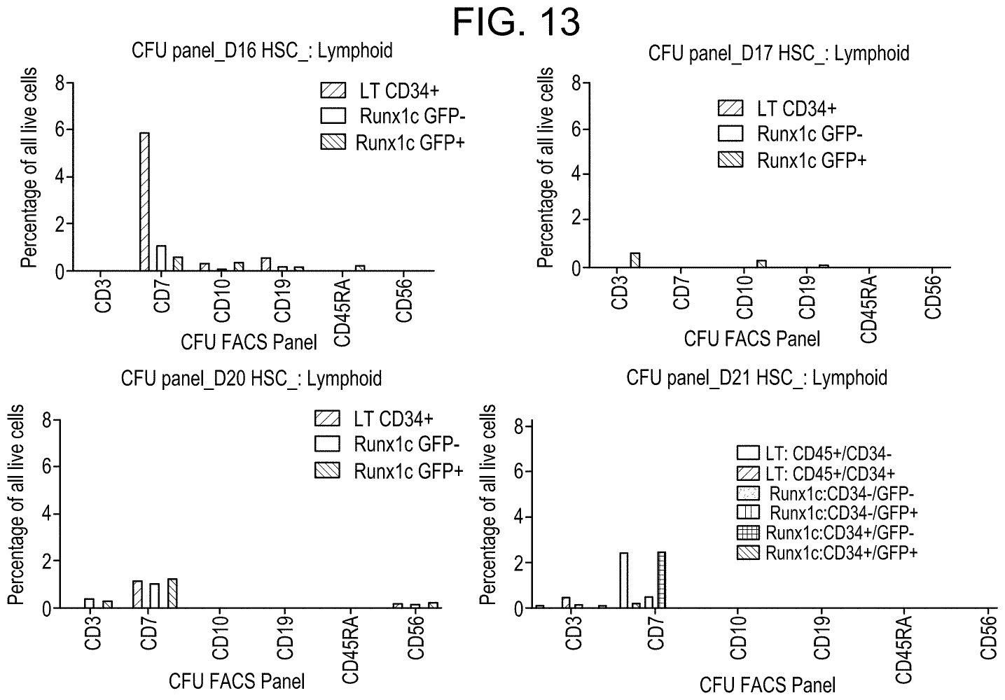

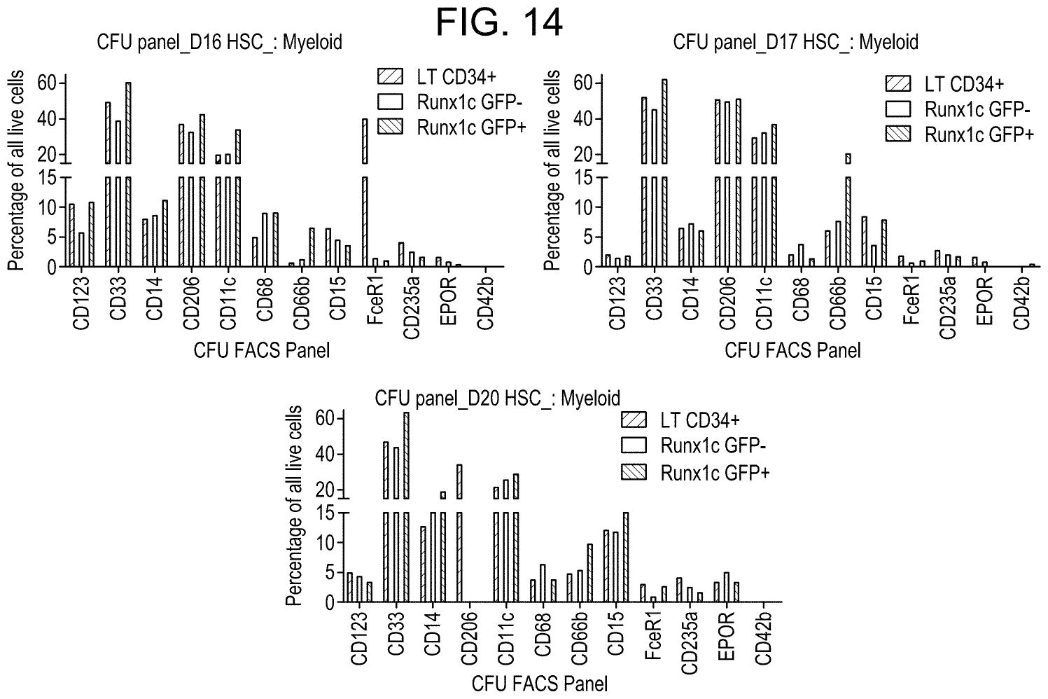

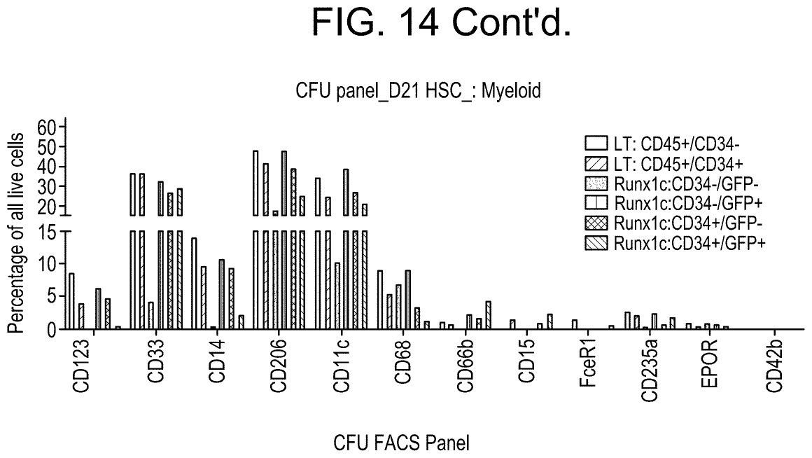

HSC cultured in MethoCult™ medium for 21 days, as described above, were harvested, titrated in single cell suspension, blocked by 1% BSA and FcR receptor blocker, stained by antibodies and FACS analysis was performed to check the expression of all lineage surface markers. FIG. 12 shows a CFU panel of common progenitor markers. HSC at day 16 which start showing strong Runx1c expression maintain several common progenitor markers after cultured into CFU. As the HSC become more mature, which shown diminished Runx1c expression in CD34+ cells, cells from CFU show minimal common progenitor markers. FIG. 13 shows a CFU panel of lymphoid markers. Although MethoCult™ was designed to expand myeloid cells in vitro, small portion of lymphoid lineage cells are identified in CFU. Include T cell, B cell and NK cells. Day 16 HSC shown to be more potent than Day 21 HSC in generating lymphoid lineage cells. FIG. 14 shows a CFU panel of myeloid markers. All stage HSC show robust potential to generating myeloid lineage cells in CFU assay. All myeloid lineage cells except platelets were identified in CFU from CD34+ HSC cells.

1. A method of producing a hematopoietic precursor cell comprising the steps of:
a) obtaining a population of pluripotent stem cells;
b) culturing the cells on day 0 in supplemented serum-free differentiated (SFD) medium under a first hypoxic condition;
c) culturing the cells in StemPro-34 medium under a second hypoxic condition;
d) culturing the cells in StemPro-34 medium under non-hypoxic conditions; and
e) culturing the cells in StemPro-34 medium under non-hypoxic expansion conditions; and
f) collect population of hematopoietic precursor cells.

2. The method of claim 1, wherein the pluripotent stem cells are human pluripotent stem cells.

3. The method of claim 1, wherein the supplemented SFD medium is supplemented with one or more of: BMP4, bFGF, Y-27632, CHIR99021, and SB-431542 added to the SFD medium.

4. The method of claim 3, wherein the BMP4 is at a concentration range of 5-25 ng/ml and added to the medium on days 0, 1, and 2; the bFGF is at a concentration range of 20-50 ng/ml and added to the medium on days 0, 1, and 2; the Y-27632 is at a concentration range of 5 μM-20 μM and added to the medium on day 0; the CHIR99021 is at a concentration range of 5 μM-20 μM and added to the medium on days 1 and 2; and the SB-431542 is at a concentration range of 0.1-20 μM and added to the medium on day 2.

5. The method of claim 4, wherein the BMP4 is at a concentration of 10 ng/ml and added to the medium on days 0, 1, and 2; the bFGF is at a concentration of 25 ng/ml and added to the medium on days 0, 1, and 2; the Y-27632 is at a concentration of 10 μM and added to the medium on day 0; the CHIR99021 is at a concentration range of 5 μM-20 μM and added to the medium on days 1 and 2; and the SB-431542 is at a concentration range of 0.1-20 μM and added to the medium on day 2.

6. The method of claim 1, wherein the StemPro-34 medium under a second hypoxic condition is supplemented with one or more of: bFGF, HSC cocktail, SB-431542, and VEGF added to the StemPro-34 medium under a second hypoxic condition.

7. The method of claim 6, wherein the bFGF is at a concentration range from 20-50 ng/ml and is added to the medium on day 3 up to day 14; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 up to day 21; the SB-431542 is at a concentration range from 0.1-20 μM and is added to the medium on day 3 to day 9; and the VEGF is at a concentration range from 20-50 ng/ml and is added to the medium on day 3 to day 14.

8. The method of claim 7, wherein the bFGF is at a concentration of 12.5 ng/ml and is added to the medium on day 3 to day 9; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 to day 9; the SB-431542 is at a concentration of 6 μM and is added to the medium on day 3; and the VEGF is at a concentration of 25 ng/ml and is added to the medium on day 3 to day 9.

9. The method of claim 1, wherein the StemPro-34 medium under non-hypoxic condition is supplemented with one or more of: bFGF, HSC cocktail, VEGF, and EHT cocktail added to the StemPro-34 medium under non-hypoxic condition.

10. The method of claim 9, wherein the bFGF is at a concentration range from 10-25 ng/ml and is added to the medium on day 3 up to day 14; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 up to day 21; the VEGF is at a concentration range from 20-50 ng/ml and is added to the medium on day 3 to day 14; and the EHT cocktail comprises BMP4, SHH, Angiotensin II, and Losartan potassium and is added to the medium on day 9 to day 14.

11. The method of claim 10, wherein the bFGF is at a concentration of 12.5 ng/ml and is added to the medium on day 9 to day 14; the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO and is added to the medium on day 6 up to day 21; the VEGF is at a concentration of 12.5 ng/ml and is added to the medium on day 9 to day 14; and the EHT cocktail comprises BMP4, SHH, Angiotensin II, and Losartan potassium and is added to the medium on day 9 to day 14.

12. The method of claim 1, wherein the StemPro-34 medium under non-hypoxic expansion condition is supplemented with HSC cocktail added to the StemPro-34 medium under non-hypoxic expansion condition.

13. The method of claim 12, wherein the HSC cocktail comprises 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO.

14. The method of claim 1, wherein the first hypoxic condition contains an 02 concentration less than 10%.

15. The method of claim 1, wherein the second hypoxic condition contains an 02 concentration less than 10%.

16. A method of producing a hematopoietic precursor cell from a pluripotent stem cell or transdifferentiation of a somatic cell, comprising culturing the pluripotent stem cell or somatic cell under conditions to generate the hematopoietic precursor cell that can differentiate into different hematopoietic lineage cells, comprising the steps of (a) obtaining a population of pluripotent stem cells, (b) inducing hematopoietic differentiation by culturing on day 0 in SFD medium, 10 uM Y-27632, 10 ng/ml BMP4 and 25 ng/ml bFGF; culturing for 1-2 days with SFD medium, 10 ng/ml BMP4, 5 ng/ml bFGF, and 8 uM CHIR99021; culturing for 1 day with StemPro34 medium, 12.5 ng/ml bFGF, and 25 ng/ml VEGF; culturing for 1-2 day with StemPro34 medium, 12.5 ng/ml bFGF, and 25 ng/ml VEGF; culturing for 2-4 days with StemPro34 medium, 12.5 ng/ml bFGF, 25 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO; culturing for 3-5 days with StemPro34 medium, 12.5 ng/ml bFGF, 12.5 ng/ml VEGF, 50 ng/ml SCF, 25 ng/ml IL-6, 25 ng/ml IL-3, 25 ng/ml FLT3L, 25 ng/ml IGF-1, 5 ng/ml IL-11, 2 U/ml EPO, 10 ng/ml BMP4, 10 ng/ml SHH, 10 ug/ml Angiotensin II, and 100 uM Losartan potassium, replaced each day; culturing for 5-10 days with StemPro34 medium, 50 ng/ml SCF, 25 ng/ml IL-6, 25 ng/ml IL-3, 25 ng/ml FLT3L, 25 ng/ml IGF-1, 5 ng/ml IL-11, and 2 U/ml EPO replaced every 3 days.

17. The method of claim 16, wherein the media with StemPro34 medium, 12.5 ng/ml bFGF, and 25 ng/ml VEGF further comprises 6 uM SB 431542.

18. The method of claim 16, wherein the media on days 2, 3, 4, or 5 further comprise 6 μm SB 431542 (TOCRIS).

19. The method of claim 1, wherein the pluripotent stem cell is capable of homing to bone marrow.

20. The method of claim 19, wherein the hematopoietic precursor cell expresses CXCR4.

21. The method of claim 1, wherein the hematopoietic precursor cell is CD34+, CD45+, CD90+, or THY1+: wherein the hematopoietic precursor cell is CD38−, Lin−, CD43− or CD73−; wherein the hematopoietic precursor cell is CD45+, CD34+, CD90+, CD38−, and Lin−: wherein the hematopoietic precursor cell is CD90+: or wherein the hematopoietic precursor cell expresses runx1c.

22-25. (canceled)

26. A hematopoietic precursor cell produced using the method of claim 1.

27. The hematopoietic precursor cell of claim 26, wherein said cell is capable of long term bone marrow engraftment.


SEQUENCE LISTING (9 sequences)

SEQ ID NO: 1 - DNA, 20 bp, Artificial Sequence
  synthetic nucleic acid
gcattttcaggaggaagcga

SEQ ID NO: 2 - DNA, 28 bp, Artificial Sequence
  synthetic nucleic acid
ctgaaagagatacatactaaagttgtcc

SEQ ID NO: 3 - DNA, 24 bp, Artificial Sequence
  synthetic nucleic acids
agtcccagaggtatccagcagagg

SEQ ID NO: 4 - DNA, 25 bp, Artificial Sequence
  synthetic nucleic acids
gtagttgccgtcgtccttgaagaag

SEQ ID NO: 5 - DNA, 25 bp, Artificial Sequence
  synthetic nucleic acids
gccatagtgaaggacagtgatggac

SEQ ID NO: 6 - DNA, 25 bp, Artificial Sequence
  synthetic nucleic acids
tcacaaacaagacagggaactggca

SEQ ID NO: 7 - DNA, 27 bp, Artificial Sequence
  synthetic nucleic acids
cagatacaatttgggtgctcaagagag

SEQ ID NO: 8 - DNA, 25 bp, Artificial Sequence
  synthetic nucleic acids
cttcttcaaggacgacggcaactac

SEQ ID NO: 9 - DNA, 25 bp, Artificial Sequence
  synthetic nucleic acids
gtccatcactgtccttcactatggc